(19) United States
(12) Patent Application Publication
(10) Pub. No.: US 2024/0224961 A1
(43) Pub. Date: Jul. 11, 2024

(54) GENETICALLY MODIFIED NON-HUMAN ANIMAL WITH HUMAN OR CHIMERIC IL1RAP

(71) Applicants: Biocytogen JiangSu Co., Ltd., Nantong (CN); Biocytogen Pharmaceuticals (Beijing) Co., Ltd., Beijing (CN)

(72) Inventors: Xiaofei Zhou, Beijing (CN); Chengzhang Shang, Beijing (CN); Chonghui Liu, Beijing (CN)

(21) Appl. No.: 18/287,574

(22) PCT Filed: May 27, 2022
(86) PCT No.: PCT/CN2022/095694

§ 371 Date: Oct. 19, 2023

(30) Foreign Application Priority Data
May 28, 2021 (CN) 202110595203.X

(51) Int. Cl. A01K 67/0275; A61K 49/00; C07K 14/715

(52) U.S. Cl. CPC A01K 67/0275; A61K 49/0008; C07K 14/7155; A01K 2207/15; A01K 2227/105; A01K 2267/03; A01K 2267/0331

(57) ABSTRACT

Provided are genetically modified animal expressing human or chimeric (e.g., humanized) IL1RAP, and methods of use thereof.

### CLAIM OF PRIORITY

This application claims the benefit of Chinese Patent Application No. CN202110595203.X, filed on May 28, 2021. The entire contents of the foregoing are incorporated herein by reference.

### TECHNICAL FIELD

This disclosure relates to a genetically modified animal expressing human or chimeric (e.g., humanized) IL1RAP, and methods of use thereof.

### BACKGROUND

Interleukin-1 receptor accessory protein (IL1RAP or IL-1RAcP) is a member of the immunoglobulin superfamily proteins consisting of soluble and membranous isoforms. IL1RAP plays an essential role in the signaling of the IL-1 family cytokines such as IL-1, IL-33 and IL-36, as well as tyrosine kinases FLT3 and C-Kit. IL1RAP generally initiates inflammatory signaling pathway through the recruitment of signaling mediators, including MYD88 and IRAK. By regulating innate and adaptive immune responses, IL1RAP plays an important role in autoimmune diseases, inflammatory responses and tumors.

Considering the important role of IL1RAP in autoimmune diseases, inflammatory responses and tumors, there is a need to develop an animal model with humanized IL1RAP.

### SUMMARY

This disclosure is related to an animal model with human IL1RAP or chimeric IL1RAP. The animal model can express human IL1RAP or chimeric IL1RAP (e.g., humanized IL1RAP) protein in its body. It can be used in the studies on the function of IL1RAP gene, and can be used in the screening and evaluation of various drugs.

In one aspect, the disclosure is related to a genetically-modified, non-human animal whose genome comprises at least one chromosome comprising a sequence encoding a human or chimeric interleukin-1 receptor accessory protein (IL1RAP).

In some embodiments, the sequence encoding the human or chimeric IL1RAP is operably linked to an endogenous regulatory element at the endogenous IL1RAP gene locus in the at least one chromosome.

In some embodiments, the sequence encoding the human or chimeric IL1RAP comprises a sequence encoding an amino acid sequence that is at least 70%, 75%, 80%, 85%, 90%, 95%, or 99% identical to SEQ ID NO: 8.

In some embodiments, the sequence encoding the human or chimeric IL1RAP comprises a sequence encoding an amino acid sequence that is at least 70%, 75%, 80%, 85%, 90%, 95%, or 99% identical to amino acids 1-367 of SEQ ID NO: 2 or amino acids 21-367 of SEQ ID NO: 2.

In some embodiments, the animal is a mammal, e.g., a monkey, a rodent or a mouse.

In some embodiments, the animal is a mouse or a rat.

In some embodiments, the animal does not express endogenous IL1RAP or expresses a decreased level of endogenous IL1RAP as compared to that of an animal without genetic modification.

In some embodiments, the animal has one or more cells expressing human or chimeric IL1RAP.

In some embodiments, the sequence encoding the human or chimeric IL1RAP comprises a part of exon 2, exon 3, exon 4, exon 5, exon 6, exon 7, exon 8 and a part of exon 9 of the human IL1RAP nucleotide sequence.

In some embodiments, the part of exon 2 comprises at least 10 bp of the human IL1RAP nucleotide sequence, and the part of exon 9 comprises at least 10 bp of the human IL1RAP nucleotide sequence.

In some embodiments, the human or chimeric IL1RAP protein comprises an amino acid sequence that is identical to SEQ ID NO: 8.

In some embodiments, the sequence encoding the human or chimeric IL1RAP comprises a nucleotide sequence that is at least 70%, 75%, 80%, 85%, 90%, 91%, 92%, 93%, 94%, 95%, 96% %, 97%, 98% or at least 99% identical to the nucleotide sequence shown in SEQ ID NO: 3, SEQ ID NO: 4, SEQ ID NO: 5, SEQ ID NO: 6, SEQ ID NO: 7, or SEQ ID NO: 10.

In some embodiments, the human or chimeric IL1RAP gene further comprises exon 1, a part of exon 2, a part of exon 9, exon 10 and exon 11 of the non-human animal's endogenous IL1RAP gene.

In one aspect, the disclosure is related to a genetically-modified, non-human animal, wherein the genome of the animal comprises a replacement of a sequence encoding a region of endogenous IL1RAP with a sequence encoding a corresponding region of human IL1RAP at an endogenous IL1RAP gene locus.

In some embodiments, the sequence encoding the corresponding region of human IL1RAP is operably linked to or under the control of an endogenous regulatory element (e.g., 5′ UTR or 3′ UTR) at the endogenous IL1RAP locus.

In some embodiments, the animal does not express endogenous IL1RAP or expresses a decreased level of endogenous IL1RAP as compared to that of an animal without genetic modification.

In some embodiments, one or more cells of the animal expresses a chimeric IL1RAP.

In some embodiments, the animal has one or more cells expressing a chimeric IL1RAP having a humanized extracellular region, transmembrane region, and/or cytoplasmic region, wherein the humanized extracellular region comprises a sequence that is at least 50%, 60%, 70%, 80%, 90%, 95%, or 99% identical to the corresponding extracellular region of human IL1RAP.

In some embodiments, the human or chimeric IL1RAP comprises a sequence that is at least 70%, 75%, 80%, 85%, 90%, 91%, 92%, 93%, 94%, 95%, 96%, 97%, 98% or at least 99% identical to SEQ ID NO: 8.

In some embodiments, the genome of the animal comprises at least SEQ ID NO: 3, SEQ ID NO: 4, SEQ ID NO: 5, SEQ ID NO: 6, SEQ ID NO: 7, SEQ ID NO: 10, or a nucleotide sequence that is at least 70%, 75%, 80%, 85%, 90%, 91%, 92%, 93%, 94%, 95%, 96%, 97%, 98% or at least 99% identical to the nucleotide sequence set forth in SEQ ID NO: 3, SEQ ID NO: 4, SEQ ID NO: 5, SEQ ID NO: 6, SEQ ID NO: 7, or SEQ ID NO: 10.

In some embodiments, the animal further comprising a deletion of one or more nucleotide from the endogenous IL1RAP gene.

In some embodiments, the animal further comprises an endogenous IL1RAP gene 5′-UTR, and/or an endogenous IL1RAP gene 3′-UTR.

In some embodiments, the animal is heterozygous or homozygous with respect to the replacement at the endogenous IL1RAP gene locus.

In one aspect, the disclosure is related to a genetically-modified, non-human animal, wherein the genome of the animal comprises an insertion of a sequence (e.g., the coding DNA sequence (CDS)) encoding a human or a chimeric IL1RAP at an endogenous IL1RAP gene locus.

In some embodiments, the inserted sequence encoding a chimeric IL1RAP includes a coding region (CDS) of the human IL1RAP gene.

In some embodiments, the inserted sequence encoding a chimeric IL1RAP includes a coding region (CDS) of the mouse IL1RAP gene.

In some embodiments, the inserted sequence is operably linked to an endogenous regulatory element at the endogenous IL1RAP locus, and one or more cells of the animal express human IL1RAP or chimeric IL1RAP.

In some embodiments, the animal does not express endogenous IL1RAP or expresses a reduced amount of endogenous IL1RAP.

In some embodiments, the inserted sequence is located immediately after 5′-UTR at the endogenous IL1RAP locus.

In some embodiments, the animal has one or more cells expressing a chimeric IL1RAP having one or more humanized extracellular regions, transmembrane regions, and cytoplasmic regions, wherein one or more of the humanized extracellular regions comprise a sequence that is at least 50%, 60%, 70%, 80%, 90%, 95%, or 99% identical to the corresponding extracellular region of human IL1RAP.

In some embodiments, one or more of the humanized extracellular regions of the chimeric IL1RAP has a sequence that has at least 10, 20, 30, 40, 50, 60, 70, 80, 90, 100, 200, 220, 240, 260, 280, 300, 320 or 340 contiguous amino acids that are identical to a contiguous sequence present in the corresponding extracellular region of human IL1RAP.

In some embodiments, the animal further comprises a deletion of one or more nucleotide from the endogenous IL1RAP gene (e.g., at exon 2).

In some embodiments, the animal is heterozygous with respect to the insertion at the endogenous IL1RAP gene locus.

In some embodiments, the animal is homozygous with respect to the insertion at the endogenous IL1RAP gene locus.

In one aspect, the disclosure is related to a method for making a genetically-modified non-human animal, the method comprising: replacing in at least one cell of the animal, at an endogenous IL1RAP gene locus, a sequence encoding a region of an endogenous IL1RAP with a sequence encoding a corresponding region of human IL1RAP.

In some embodiments, the sequence encoding the corresponding region of human IL1RAP gene comprises exon 1, exon 2, exon 3, exon 4, exon 5, exon 6, exon 7, exon 8, exon 9, exon 10, and/or exon 11, or a part thereof, of a human IL1RAP gene.

In some embodiments, the sequence encoding the corresponding region of human IL1RAP gene encodes a sequence that is at least 90% identical to amino acids 1-367 of SEQ ID NO: 2 or amino acids 21-367 of SEQ ID NO:2.

In some embodiments, the sequence encoding the corresponding region of human IL1RAP gene is at least 90% identical to SEQ ID NO: 5.

In some embodiments, the sequence encoding a region of human IL1RAP gene is identical to SEQ ID NO: 5.

In some embodiments, the method further comprises deleting one or more nucleotides of the endogenous IL1RAP gene.

In some embodiments, a part of exon 2, all of exon 3, exon 4, exon 5, exon 6, exon 7, exon 8, and a part of exon 9 of the endogenous IL1RAP are replaced.

In some embodiments, the sequence encoding a region of the endogenous IL1RAP is replaced by a part of exon 2, all of exon 3, exon 4, exon 5, exon 6, exon 7, exon 8, and a part of exon 9 of the human IL1RAP gene.

In some embodiments, the part of exon 2 of the human IL1RAP gene comprises at least 10 bp contiguous human nucleotides, wherein the part of exon 9 of the human IL1RAP gene comprises at least 10 bp contiguous human nucleotides.

In one aspect, the disclosure is related to a method for making a genetically-modified, non-human animal, comprising: inserting in at least one cell of the animal, at an endogenous IL1RAP gene locus, a sequence encoding a region of human IL1RAP gene.

In some embodiments, the sequence encoding the region of human IL1RAP gene comprises a part of exon 2, exon 3, exon 4, exon 5, exon 6, exon 7, exon 8, and a part of exon 9 of a human IL1RAP gene.

In some embodiments, the sequence encoding a region of human IL1RAP gene encodes a sequence that is at least 90% identical to SEQ ID NO: 2.

In some embodiments, the animal is a mouse, and the endogenous IL1RAP locus is within exon 2 of the mouse IL1RAP gene.

In some embodiments, the method further comprises deleting one or more nucleotides of the endogenous IL1RAP gene.

In one aspect, the disclosure is related to a non-human animal comprising at least one cell comprising a nucleotide sequence encoding a humanized IL1RAP polypeptide, wherein the humanized IL1RAP polypeptide comprises at least 50 contiguous amino acid residues that are identical to the corresponding contiguous amino acid sequence of a human IL1RAP, wherein the animal expresses the humanized IL1RAP.

In some embodiments, the humanized IL1RAP polypeptide has at least 50 contiguous amino acid residues that are identical to the corresponding contiguous amino acid sequence of a human IL1RAP extracellular region.

In some embodiments, the humanized IL1RAP polypeptide comprises a sequence that is at least 90%, 95%, or 99% identical to amino acids 1-367 of SEQ ID NO: 2 or amino acids 21-367 of SEQ ID NO: 2.

In some embodiments, the nucleotide sequence is operably linked to an endogenous IL1RAP regulatory element of the animal (e.g., 5′-UTR).

In some embodiments, the humanized IL1RAP polypeptide comprises a humanized extracellular region, an endogenous IL1RAP transmembrane region and/or an endogenous IL1RAP cytoplasmic region.

In some embodiments, the nucleotide sequence is integrated to an endogenous IL1RAP gene locus of the animal.

In one aspect, the disclosure is related to a method of making a genetically-modified mouse cell that expresses a chimeric IL1RAP, the method comprising: replacing at an endogenous mouse IL1RAP gene locus, a nucleotide sequence encoding a region of mouse IL1RAP with a nucleotide sequence encoding a corresponding region of human IL1RAP, thereby generating a genetically-modified mouse cell that includes a nucleotide sequence that encodes the chimeric IL1RAP, wherein the mouse cell expresses the chimeric IL1RAP.

In some embodiments, the chimeric IL1RAP polypeptide comprises a sequence that is at least 90%, 95%, or 99% identical to amino acids 1-367 of SEQ ID NO: 2 or amino acids 21-367 of SEQ ID NO: 2.

In some embodiments, the chimeric IL1RAP comprises: the extracellular region of human or humanized IL1RAP; and the transmembrane region and/or the cytoplasmic region of mouse IL1RAP.

In some embodiments, the animal further comprises a sequence encoding an additional human or chimeric protein.

In some embodiments, the additional human or chimeric protein is IL-1, IL-1 receptor, PD-1, PD-L1, IL6, TNF, 41BB, CD40, IL17, TNFR2, IL4, IL33, TIGIT, OX40, IL1B, IL1R1, IL13, CCR5, CCR8 or IL10.

In some embodiments, the animal or mouse further comprises a sequence encoding an additional human or chimeric protein.

In some embodiments, the additional human or chimeric protein is IL-1, IL-1 receptor, PD-1, PD-L1, IL6, TNF, 41BB, CD40, IL17, TNFR2, IL4, IL33, TIGIT, OX40, ILIB, IL1R1, IL13, CCR5, CCR8 or IL10.

In one aspect, the disclosure is related to a method of determining effectiveness of a therapeutic agent targeting IL1RAP for the treatment of an immune-related disease, the method comprising: administering the therapeutic agent targeting IL1RAP to the animal described herein, wherein the animal has an immune-related disease; and determining the effects of the therapeutic agent targeting IL1RAP to the immune-related disease of the animal.

In some embodiments, the immune-related disease is an autoimmune disease.

In some embodiments, determining the effects of the therapeutic agent targeting IL1RAP to the immune-related disease of the animal comprises measuring the T cell proliferation, cytokine secretion and killing capacity.

In some embodiments, determining the effects of the therapeutic agent targeting IL1RAP to the immune-related disease of the animal further comprises assessing individual body weight, fat mass, activation pathway, neuroprotective activity, or metabolic changes, and the metabolic changes include changes in food consumption or water consumption.

In one aspect, the disclosure is related to a method of determining effectiveness of a human or humanized antibody for the treatment of a disease, the method comprising administering the antibody to the animal described herein; and determining the effects of the antibody on the disease.

In some embodiments, the disease is a tumor.

In some embodiments, the animal further comprises a sequence encoding a human or chimeric IL-1, IL-1 receptor, PD-1, PD-L1, IL6, TNF, 41BB, CD40, IL17, TNFR2, IL4, IL33, TIGIT, OX40, IL1B, IL1R1, IL13, CCR5, CCR8 or IL10.

In some embodiments, the antibody is an anti-IL1RAP antibody.

In one aspect, the disclosure is related to a method of determining effectiveness of an anti-IL1RAP antibody for the treatment of cancer, the method comprising: administering the anti-IL1RAP antibody to the animal described herein, wherein the animal has a tumor; and determining the inhibitory effects of the anti-IL1RAP antibody to the tumor.

In some embodiments, the tumor comprises one or more cancer cells that are injected into the animal.

In some embodiments, determining the inhibitory effects of the anti-IL1RAP antibody to the tumor comprises measuring the tumor volume in the animal.

In some embodiments, the tumor cells are derived from non-small cell lung cancer (NSCLC), pancreatic cancer, breast cancer, colorectal cancer, liver cancer, esophageal cancer, head and neck cancer, bladder cancer, or colon cancer.

In one aspect, the disclosure is related to a protein comprising an amino acid sequence, wherein the amino acid sequence is one of the following:
an amino acid sequence set forth in SEQ ID NO: 8;
an amino acid sequence that is at least 90% identical to SEQ ID NO: 8;
an amino acid sequence that is at least 91%, 92%, 93%, 94%, 95%, 96%, 97%, 98%, or 99% identical to SEQ ID NO: 8;
an amino acid sequence that is different from the amino acid sequence set forth in SEQ ID NO: 8 by no more than 10, 9, 8, 7, 6, 5, 4, 3, 2 or 1 amino acid; and
an amino acid sequence that comprises a substitution, a deletion and/or insertion of one, two, three, four, five or more amino acids to the amino acid sequence set forth in SEQ ID NO: 8.

In one aspect, the disclosure is related to a nucleic acid comprising a nucleotide sequence, wherein the nucleotide sequence is one of the following:
a sequence that encodes the protein described herein;
SEQ ID NO: 5;
SEQ ID NO: 7;
SEQ ID NO: 10;
a sequence that is at least 90% identical to SEQ ID NO: 5, SEQ ID NO: 7, SEQ ID NO: 10;
a sequence that is at least 91%, 92%, 93%, 94%, 95%, 96%, 97%, 98%, or 99% identical to SEQ ID NO: 5, or SEQ ID NO: 7, SEQ ID NO: 10.

In one aspect, the disclosure is related to a cell comprising the protein described herein and/or the nucleic acid described herein.

In one aspect, the disclosure is related to an animal comprising the protein described herein and/or the nucleic acid described herein.

In one aspect, the disclosure is related to a method of determining toxicity of an anti-IL1RAP antibody, the method comprising administering the anti-IL1RAP antibody to the animal described herein; and determining weight change of the animal.

In some embodiments, the method further comprising performing a blood test (e.g., determining red blood cell count).

In addition, the animal models prepared by the methods described herein can be used in drug screening, pharmacodynamics studies, testing treatments for IL1RAP related diseases. The disclosure also provides a powerful tool for studying the function of IL1RAP protein and a platform for screening drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIG. 1 is a schematic diagram of the comparison between mouse IL1RAP gene and human IL1RAP gene locus (not to scale).

FIG. 2 is a schematic diagram of the humanization of mouse IL1RAP gene (not to scale). A part of exon 2, exon 3, exon 4, exon 5, exon 6, exon 7, exon 8 and a part of exon 9 of human IL1RAP gene was used to replace the corresponding sequence mouse in the mouse IL1RAP gene.

FIG. 3 is a schematic diagram of the humanization of mouse IL1RAP gene (not to scale). A human-mouse hybrid sequence was inserted or replaced into a specific location in the mouse IL1RAP gene.

FIG. 4 is a schematic diagram of IL1RAP gene targeting strategy and targeting vector design (not to scale).

FIG. 5 shows the Southern Blot results. WT means wild type control.

FIGS. 6A-6B show the PCR genotype identification results of IL1RAP humanized mice (F1 generation) prepared according to Example 1. WT means wild type control. H₂O means water control. PC means positive control. M means marker.

FIG. 7 shows the detection results of humanized IL1RAP mRNA by RT-PCR. +/+ is a wild-type C57BL/6 mouse. H/H is a IL1RAP humanized homozygous mouse. H₂O means water control.

FIG. 8 shows the alignment between mouse IL1RAP amino acid sequence (NP_032390.1; SEQ ID NO: 1) and human IL1RAP amino acid sequence (NP_001161401.1; SEQ ID NO: 2).

FIG. 9 shows the alignment between rat IL1RAP amino acid sequence (NP_037100.1; SEQ ID NO: 36) and human IL1RAP amino acid sequence (NP_001161401.1; SEQ ID NO: 2).

### DETAILED DESCRIPTION

This disclosure relates to transgenic non-human animal with human or chimeric (e.g., humanized) IL1RAP, and methods of use thereof.

The present disclosure provides a genetically modified animal expressing human or chimeric (e.g., humanized) IL1RAP, and methods of use thereof. The humanized animal models have various important applications. For example, due to the presence of human or humanized genes, the animals can express or express in part of the proteins with human functions, so as to greatly reduce the differences in clinical trials between humans and animals, and provide the possibility of drug testing at animal levels.

### IL1RAP

Cytokines such as interleukin (IL)-1 are regarded as the counterparts of the hormones in the endocrine system. These proteins/glycoproteins play a substantial role in the control of immune responses, including activation and inhibition of the inflammatory cascades, as well as triggering the proliferation and differentiation of various cells. Their selective binding to specific receptors usually leads to signal transduction and distinct biological consequences. The IL-1 receptor accessory protein (IL1RAcP), also known as IL1RAP or IL-1R3, is a co-receptor involved in the molecular structure of some determined cytokine receptors, including IL-1, IL-33, IL-36, as well as tyrosine kinases, including Fms related receptor tyrosine kinase 3 (FLT3) and C-kit.

IL1RAP exists in both soluble and membranous forms with different functionalities. The soluble form of IL1RAP (sIL1RAP) in the serum, unlike its membranous form (mIL1RAP) on various cells, shows anti-inflammatory properties, and measurement of its concentration in blood has been suggested as a criterion for the monitoring of certain inflammatory diseases. In addition, IL1RAP plays a critical role in the development of systemic inflammations and its complications. Moreover, its application as a therapeutic target was investigated in animal models of inflammatory diseases, such as peritonitis, psoriasis, and airway inflammation. Furthermore, antibody-mediated targeting of IL1RAP could result in a remarkable decrease in inflammation and may even lead to remission in animal models.

IL1RAP is a member of the immunoglobulin (Ig) superfamily of proteins, and is composed of three extracellular Ig domains and a cytoplasmic domain called Toll/IL-1 receptor (TIR). IL1RAP plays a critical role in IL-1 receptor-I signaling, and its internalization is necessary for initiating the signaling process, but this is not sufficient and a set of molecules, including IL-1 receptor I, IL1RAP, and IL-1 (α or β), are needed to complete the initiation of the signaling cascade. When IL-1, IL-33 and IL-36 bind to their receptors, they can lead to IL1RAP aggregation and the recruitment of the adaptor protein MyD88, which interacts via its carboxy-terminal TIR domain. The IL-1 receptor-associated kinase (IRAK) is another protein involved in this signaling, and the intracellular domains of both IL-1 receptor I and IL1RAP are required to recruit the IRAK molecule. This leads to phosphorylation IRAK and dissociation from IL-1R. Interaction of IRAK with TRAF6 promotes further activation of transcription factors nuclear factor kappa B (NF-κB) and N-terminal region of Janus kinase (JAK).

Up to now, four isoforms of the IL1RAP have been identified, and their signaling pathways were studied. The membranous form, namely, mIL1RAP, plays an influential role in receptor-mediated signaling of IL-1, IL-33, IL-36, as well as FLT3 and C-Kit in AML cells. The role of C-Kit and FLT3, has been previously established in tumor growth and progression including angiogenesis, cell proliferation, and cell migration. SCF (C-Kit Ligand) and FLT3 ligand bind to c-Kit and FLT3, respectively, causing receptor homodimerization and autophosphorylation as well as activation of various signaling pathways. Janus kinase (JAK) interacts with phosphorylated FLT-3 and c-Kit to activate various STAT molecules as transcription factors. Activation of PI3-kinase, Src, and the small G protein Ras activates a variety of pathways, including MAP-kinase and Akt/PKB.

An alternative RNA splicing of IL1RAP gene can develop a secretory isoform that lacks the intracellular domain, resulting in a soluble protein. The secretory form can bind to soluble IL-1 receptor II in plasma and increase its affinity to both IL-1α and IL-1β. IL-1 receptor II acts as a natural regulatory protein in the IL-1 signaling pathway due to ineffective signal transduction. In the absence of the cytoplasmic TIR domain, IL-1 receptor, acts as a decoy receptor for IL-1 and based on its interaction with IL-1 receptor II, it might act as an anti-inflammatory factor. The third isoform, known as sIL1RAP-β, is also a secretory molecule and shows a similar functionality as mentioned for sIL1RAcP. Structurally, this isoform is distinguishable from the sIL1RAP based on a shorter third Ig domain, however, similar to SIL1RAP, it can contribute to the regulation of the inflammation induced by IL-1. The fourth isoform, known as AcPb, is exclusively expressed in CNS neurons, and its expression level in CNS is much higher than other isoforms. It is noteworthy to mention that the bronchial epithelial cells are the only non-neuronal cells with the AcPb mRNA.

A detailed review of IL1RAP and its functions can be found in Zarezadeh Mehrabadi, Ali, et al. “The roles of interleukin-1 receptor accessory protein in certain inflammatory conditions.” Immunology 166.1 (2022): 38-46, which is incorporated by reference in its entirety.

The mouse IL1RAP gene (Gene ID: 16180) is located in Chromosome 16 of the mouse genome, and corresponds to nucleotide 26400259 to 26548878 of NC_000082.7 (GRCm39 (GCF_000001635.27)). The 5′-UTR is from 26400507 to 26400636 and 26442905, exon 1 is from 26400507 to 26400636, the first intron is from 26400637 to 26442904, exon 2 is from 26442905 to 26442969, the second intron is from 26442970 to 26495458, exon 3 is from 26495459 to 26495744, the third intron is from 26495745 to 26498852, exon 4 is from 26498853 to 26499039, the fourth intron is from 26499040 to 26511494, exon 5 is from 26511495 to 26511660, the fifth intron is from 26511661 to 26513971, exon 6 is from 26513972 to 26514043 the sixth intron is from 26514044 to 26517576, exon 7 is from 26517577 to 26517703, the seventh intron is from 26517704 to 26519837, exon 8 is from 26519838 to 26519986, the eighth intron is from 26519987 to 26529230, exon 9 is from 26529231 to 26529380, the ninth intron is from 26529381 to 26530866, exon 10 is from 26530867 to 26531010, the tenth intron is from 26531011 to 26533450, exon 11 is from 26533451 to 26536494, and the 3′-UTR is from 26533819 to 26536494, based on transcript NM_008364.2. All relevant information for mouse IL1RAP locus can be found in the NCBI website with Gene ID: 16180, which is incorporated by reference herein in its entirety. The location for each exon and each region in mouse IL1RAP nucleotide sequence and amino acid sequence is listed below:

TABLE 1

| Mouse IL1RAP | | NP_032390.1 |
|---|---|---|
| (approximate | NM_008364.2 | 570 aa |
| location) | 4576 bp | (SEQ ID NO: 1) |
| Exon 1 | 1-182 | Non-coding |
| Exon 2 | 183-247 | 1-21 |
| Exon 3 | 248-533 | 22-117 |
| Exon 4 | 534-720 | 118-179 |
| Exon 5 | 721-886 | 180-234 |
| Exon 6 | 887-958 | 235-258 |
| Exon 7 | 959-1085 | 259-301 |
| Exon 8 | 1086-1234 | 302-350 |
| Exon 9 | 1235-1384 | 351-400 |
| Exon 10 | 1385-1528 | 401-448 |
| Exon 11 | 1529-4576 | 449-570 |
| Signal peptide | 184-243 | 1-20 |
| Extracellular | 244-1284 | 21-367 |
| Transmembrane | 1285-1347 | 368-388 |
| Cytoplasmic | 1348-1893 | 389-570 |
| Replaced mouse | 184-1284 | 1-367 |
| sequence | | |
| Remaining mouse | 1285-1896 | 368-570 |
| sequence | | |

The human IL1RAP gene (Gene ID: 3556) is located in Chromosome 3 of the human genome, which is located from 190514085 to 190659750 of NC_000003.12 (GRCh38.p14 (GCF_000001405.40)). The S′-UTR is from 190,514,051 to 190,514,219 and 190,564,289 to 190,564,290, exon1 is from 190,514,051 to 190,514,219, the first intron is from 190,514,220 to 190,564,288, exon2 is from 190,564,289 to 190,564,353, the second intron is from 190,564,354 to 190,604,127 exon3 is from 190,604,128 to 190,604,413, the third intron is from 190,604,414 to 190,608,994, exon4 is from 190,608,995 to 190,609,181, the fourth intron is from 190,609,182 to 190,620,274, exon5 is from 190,620,275 to 190,620,440, the fifth intron is from 190,620,441 to 190,623,343, exon6 is from 190,623,344 to 190,623,415 the sixth intron is from 190,623,416 to 190,627,322, exon7 is from 190,627,323 to 190,627,449, the seventh intron is from 190,627,450 to 190,629,349, exon8 is from 190,629,350 to 190,629,498, the eighth intron is from 190,629,499 to 190,644,247, exon9 is from 190,644,248 to 190,644,397, the ninth intron is from 190,644,398 to 190,645,698, exon10 is from 190,645,699 to 190,645,842, the tenth intron is from 190,645,843 to 190,648,337, exon11 is from 190,648,338 to 190,651,519, the 3′-UTR is from 190,648,706 to 190,651,519, based on transcript NM_213602.3. All relevant information for human IL1RAP locus can be found in the NCBI website with Gene ID: 3556, which is incorporated by reference herein in its entirety. The location for each exon and each region in the human IL1RAP nucleotide sequence and amino acid sequence is listed below:

TABLE 2

| Human IL1RAP | | NP_001161401.1 |
|---|---|---|
| (approximate | NM_001167929.2 | 570 aa |
| location) | 4658 bp | SEQ ID NO: 2 |
| Exon 1 | 1-135 | Non-coding |
| Exon 2 | 136-200 | 1-21 |
| Exon 3 | 201-486 | 22-117 |
| Exon 4 | 487-673 | 118-179 |
| Exon 5 | 674-839 | 180-234 |
| Exon 6 | 840-911 | 235-258 |
| Exon 7 | 912-1038 | 259-301 |
| Exon 8 | 1039-1187 | 302-350 |
| Exon 9 | 1188-1337 | 351-400 |
| Exon 10 | 1338-1481 | 401-448 |
| Exon 11 | 1482-4658 | 449-570 |
| Signal peptide | 137-196 | 1-20 |
| Extracellular | 197-1237 | 21-367 |
| Transmembrane | 1238-1300 | 368-388 |
| Cytoplasmic | 1301-1846 | 389-570 |
| Donor region | 137-1237 | 1-367 |
| in Example | | |

IL1RAP genes, proteins, and locus of the other species are also known in the art. For example, the gene ID for IL1RAP in Rattus norvegicus is 25466, the gene ID for IL1RAP in Macaca mulatta (Rhesus monkey) is 574387, the gene ID for IL1RAP in Canis lupus familiaris (dog) is 488126, the gene ID for IL1RAP in Pan troglodytes (chimpanzee) is 460935, the gene ID for IL1RAP in Bos taurus (cattle) is 539334, and the gene ID for IL1RAP in Gallus gallus (chicken) is 424908. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) IL1RAP nucleotide sequence and/or amino acid sequences. In some embodiments, the entire sequence of mouse exon 1, exon 2, exon 3, exon 4, exon 5, exon 6, exon 7, exon 8, exon 9, exon 10, exon 11, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, exon 9, exon 10, exon 11, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, 600, 700, 800, 900, 1000, 1500, 2000, 2500, 3000, 3500, 4000, or 4500 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 220, 240, 260, 280, 300, 320, 340, 360, 380, 400, 420, 440, 460, 480, 500, 520, 540, 560, or 570 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, exon 10, exon 11, signal peptide, extracellular region, transmembrane region, or cytoplasmic region. In some embodiments, a region, a portion, or the entire sequence of mouse exon 1, exon 2, exon 3, exon 4, exon 5, exon 6, exon 7, exon 8, exon 9, exon 10, and/or exon 11 are replaced by the human exon 1, exon 2, exon 3, exon 4, exon 5, exon 6, exon 7, exon 8, exon 9, exon 10, and/or exon 11 sequence.

In some embodiments, the present disclosure also provides a chimeric (e.g., humanized) IL1RAP nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL1RAP gene, mouse IL1RAP amino acid sequence (e.g., SEQ ID NO: 1), or a portion thereof (e.g., exon 1, exon 2, exon 3, exon 4, exon 5, exon 6, exon 7, exon 8, exon 9, exon 10, and/or exon 11); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL1RAP gene sequence, human IL1RAP amino acid sequence (e.g., SEQ ID NO: 2), or a portion thereof (e.g., exon 1, exon 2, exon 3, exon 4, exon 5, exon 6, exon 7, exon 8, exon 9, exon 10, and/or exon 11).

In some embodiments, the nucleic acids as described herein are operably linked to a promotor or regulatory element, e.g., an endogenous mouse IL1RAP promotor, an inducible promoter, an enhancer, and/or mouse or human regulatory elements.

In some embodiments, the nucleic acids as described herein are operably linked to a Woodchuck Hepatitis Virus (WHP) Posttranscriptional Regulatory Element (WPRE). In some embodiments, the nucleic acids as described herein are operably linked to a poly A (polyadenylation) signal sequence.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 250, 300, 350, 400, 500, 600, 700, 800, 900, 1000, 1500, 2000, 2500, 3000, 3500, 4000, or 4500 nucleotides, e.g., contiguous or non-contiguous nucleotides) that are different from a portion of or the entire mouse IL1RAP nucleotide sequence (e.g., exon 1, exon 2, exon 3, exon 4, exon 5, exon 6, exon 7, exon 8, exon 9, exon 10, and/or exon 11).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 250, 300, 350, 400, 500, 600, 700, 800, 900, 1000, 1500, 2000, 2500, 3000, 3500, 4000, or 4500 nucleotides, e.g., contiguous or non-contiguous nucleotides) that is the same as a portion of or the entire mouse IL1RAP nucleotide sequence (e.g., exon 1, exon 2, exon 3, exon 4, exon 5, exon 6, exon 7, exon 8, exon 9, exon 10, exon 11).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 250, 300, 350, 400, 500, 600, 700, 800, 900, 1000, 1500, 2000, 2500, 3000, 3500, 4000, or 4500 nucleotides, e.g., contiguous or non-contiguous nucleotides) that is different from a portion of or the entire human IL1RAP nucleotide sequence (e.g., exon 1, exon 2, exon 3, exon 4, exon 5, exon 6, exon 7, exon 8, exon 9, exon 10, exon 11).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 250, 300, 350, 400, 500, 600, 700, 800, 900, 1000, 1500, 2000, 2500, 3000, 3500, 4000, or 4500 nucleotides, e.g., contiguous or non-contiguous nucleotides) that is the same as a portion of or the entire human IL1RAP nucleotide sequence (e.g., exon 1, exon 2, exon 3, exon 4, exon 5, exon 6, exon 7, exon 8, exon 9, exon 10, exon 11).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 220, 240, 260, 280, 300, 320, 340, 360, 380, 400, 420, 440, 460, 480, 500, 520, 540, 560, or 570 amino acid residues, e.g., contiguous or non-contiguous amino acid residues) that is different from a portion of or the entire mouse IL1RAP amino acid sequence (e.g., exon 1, exon 2, exon 3, exon 4, exon 5, exon 6, exon 7, exon 8, exon 9, exon 10, exon 11 or SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 220, 240, 260, 280, 300, 320, 340, 360, 380, 400, 420, 440, 460, 480, 500, 520, 540, 560, or 570 amino acid residues, e.g., contiguous or non-contiguous amino acid residues) that is the same as a portion of or the entire mouse IL1RAP amino acid sequence (e.g., exon 1, exon 2, exon 3, exon 4, exon 5, exon 6, exon 7, exon 8, exon 9, exon 10, exon 11 or SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 220, 240, 260, 280, 300, 320, 340, 360, 380, 400, 420, 440, 460, 480, 500, 520, 540, 560, or 570 amino acid residues, e.g., contiguous or non-contiguous amino acid residues) that is different from a portion of or the entire human IL1RAP amino acid sequence (e.g., exon 1, exon 2, exon 3, exon 4, exon 5, exon 6, exon 7, exon 8, exon 9, exon 10, exon 11, or SEQ ID NO: 2).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 220, 240, 260, 280, 300, 320, 340, 360, 380, 400, 420, 440, 460, 480, 500, 520, 540, 560, or 570 amino acid residues, e.g., contiguous or non-contiguous amino acid residues) that is the same as a portion of or the entire human IL1RAP amino acid sequence (e.g., exon 1, exon 2, exon 3, exon 4, exon 5, exon 6, exon 7, exon 8, exon 9, exon 10, exon 11, or SEQ ID NO: 2).

The present disclosure also provides a humanized IL1RAP mouse amino acid sequence, wherein the amino acid sequence is selected from the group consisting of:
a) an amino acid sequence shown in SEQ ID NO: 8;
b) an amino acid sequence having a homology of at least 90% with or at least 90% identical to the amino acid sequence shown in SEQ ID NO: 8;
c) an amino acid sequence encoded by a nucleic acid sequence, wherein the nucleic acid sequence is able to hybridize to a nucleotide sequence encoding the amino acid shown in SEQ ID NO: 8 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 8;
e) an amino acid sequence that is different from the amino acid sequence shown in SEQ ID NO: 8 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 8.

The present disclosure also relates to a IL1RAP nucleic acid (e.g., DNA, RNA or mRNA) sequence, wherein the nucleic acid sequence can be selected from the group consisting of:
a) a nucleic acid sequence as shown in SEQ ID NO: 7, or a nucleic acid sequence encoding a homologous IL1RAP amino acid sequence of a humanized mouse;
b) a nucleic acid sequence that is shown in SEQ ID NO: 7;
c) a nucleic acid sequence that is able to hybridize to the nucleotide sequence as shown in SEQ ID NO: 7 under a low stringency condition or a strict stringency condition;
d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 7;
e) a nucleic acid sequence that encodes an amino acid sequence, wherein the amino acid sequence has a homology of at least 90% with or at least 90% identical to the amino acid sequence shown in SEQ ID NO: 8;
f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 8;
g) a nucleic acid sequence that encodes an amino acid sequence, wherein the amino acid sequence is different from the amino acid sequence shown in SEQ ID NO: 8 by no more than 10, 9, 8, 7, 6, 5, 4, 3, 2 or no more than 1 amino acid; and/or
h) a nucleic acid sequence that encodes an amino acid sequence, wherein the amino acid sequence comprises a substitution, a deletion and/or insertion of one or more amino acids to the amino acid sequence shown in SEQ ID NO: 8.

The present disclosure further relates to a IL1RAP genomic DNA sequence of a humanized mouse. The DNA sequence is obtained by a reverse transcription of the mRNA sequence homologous to the sequence shown in SEQ ID NO: 7.

The disclosure also provides an amino acid sequence that has a homology of at least 90% with, or at least 90% identical to the sequence shown in SEQ ID NO: 8, and has protein activity. In some embodiments, the homology with the sequence shown in SEQ ID NO: 8 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 8 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 7, and encodes a polypeptide that has protein activity. In some embodiments, the homology with the sequence shown in SEQ ID NO: 7 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) IL1RAP from an endogenous IL1RAP locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous IL1RAP locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wildtype amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized IL1RAP gene or a humanized IL1RAP nucleic acid. In some embodiments, at least one or more portions of the gene or the nucleic acid is from the human IL1RAP gene, at least one or more portions of the gene or the nucleic acid is from a non-human IL1RAP gene. In some embodiments, the gene or the nucleic acid comprises a sequence that encodes a IL1RAP protein. The encoded IL1RAP protein is functional or has at least one activity of the human IL1RAP protein or the non-human IL1RAP protein, e.g., binding with IL-1 receptor.

In some embodiments, the chimeric protein or the chimeric polypeptide is a humanized IL1RAP protein or a humanized IL1RAP polypeptide. In some embodiments, at least one or more portions of the amino acid sequence of the protein or the polypeptide is from a human IL1RAP protein, and at least one or more portions of the amino acid sequence of the protein or the polypeptide is from a non-human IL1RAP protein. The humanized IL1RAP protein or the humanized IL1RAP polypeptide is functional or has at least one activity of the human IL1RAP protein or the non-human IL1RAP protein.

In some embodiments, one or more of the three extracellular Ig domains of the IL1RAP protein are human or humanized. In some embodiments, the first extracellular Ig domain of the IL1RAP protein is human or humanized. In some embodiments, the second extracellular Ig domain of the IL1RAP protein is human or humanized. In some embodiments, the third extracellular Ig domain of the IL1RAP protein is human or humanized.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized IL1RAP animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Ry knockout mice, NOD/SCID/γc^{null} mice (Ito, M. et al., NOD/SCID/γc^{null} mouse: an excellent recipient mouse model for engraftment of human cells, Blood 100 (9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human IL1RAP locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Ry knockout mice, NOD/SCID/γc^{null} mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of IL1RAP coding sequence with the corresponding human IL1RAP coding sequence or an insertion of human IL1RAP coding sequence or chimeric IL1RAP coding sequence.

Genetically modified non-human animals that comprise a modification of an endogenous non-human IL1RAP locus. In some embodiments, the modification can comprise a human nucleic acid sequence encoding at least a portion of a IL1RAP protein (e.g., at least 10%, 20%, 30%, 40%, 50%, 60%, 70%, 80%, 90%, 95%, 96%, 97%, 98%, or 99% identical to the IL1RAP protein sequence). Although genetically modified cells are also provided that can comprise the modifications described herein (e.g., ES cells, somatic cells), in many embodiments, the genetically modified non-human animals comprise the modification of the endogenous IL1RAP locus in the germline of the animal.

Genetically modified animals can express a human IL1RAP and/or a chimeric (e.g., humanized) IL1RAP from endogenous loci, wherein the endogenous mouse IL1RAP gene has been replaced with a human IL1RAP gene and/or a nucleotide sequence that encodes a region of human IL1RAP sequence or an amino acid sequence that is at least 10%, 20%, 30%, 40%, 50%, 60%, 70%, 80%, 90%, 95%, 96%, 97%, 98%, or 99% identical to the human IL1RAP sequence. In various embodiments, an endogenous non-human IL1RAP locus is modified in whole or in part to comprise human nucleic acid sequence encoding at least one protein-coding sequence of a IL1RAP protein.

In some embodiments, the genetically modified animals (e.g., mice) can express a human IL1RAP and/or a chimeric (e.g., humanized) IL1RAP.

In some embodiments, the genetically modified mice express the human IL1RAP and/or chimeric IL1RAP (e.g., humanized IL1RAP) from endogenous loci that are under control of mouse promoters and/or mouse regulatory elements. The replacement (s) at the endogenous mouse loci provide non-human animals that express human IL1RAP or chimeric IL1RAP (e.g., humanized IL1RAP) in appropriate cell types and in a manner that does not result in the potential pathologies observed in some other transgenic mice known in the art. The human IL1RAP or the chimeric IL1RAP (e.g., humanized IL1RAP) expressed in animal can maintain one or more functions of the wildtype mouse or human IL1RAP in the animal. For example, human or non-human IL1RAP ligands (e.g., IL-1 receptor) can bind to the expressed IL1RAP. Furthermore, in some embodiments, the animal does not express endogenous IL1RAP. As used herein, the term “endogenous IL1RAP” refers to IL1RAP protein that is expressed from an endogenous IL1RAP nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL1RAP (SEQ ID NO: 2).

The genome of the genetically modified animal can comprise a replacement at an endogenous IL1RAP gene locus of a sequence encoding a region of endogenous IL1RAP with a sequence encoding a corresponding region of human IL1RAP. In some embodiments, the sequence that is replaced is any sequence within the endogenous IL1RAP gene locus, e.g., exon 1, exon 2, exon 3, exon 4, exon 5, exon 6, exon 7, exon 8, exon 9, exon 10, exon 11, 5′-UTR, 3′-UTR, the first intron, the second intron, the third intron, the fourth intron, the fifth intron, the sixth intron, the extracellular region, the cytoplasmic region, etc. In some embodiments, the sequence that is replaced is a part of exon 2, exon 3, exon 4, exon 5, exon 6, exon 7, exon 8 and a part of exon 9 of the endogenous IL1RAP gene.

In some embodiments, a sequence that encodes an amino acid sequence (e.g., human IL1RAP or chimeric IL1RAP) is inserted after 5′-UTR (e.g., immediately after 5′-UTR), or immediately before the start codon (e.g., within 10, 20, 30, 40, 50, 60, 70, 80, 90, or 100 nucleic acids). The start codon is the first codon of a messenger RNA (mRNA) transcript translated by a ribosome. The start codon always codes for methionine in eukaryotes and a modified Met (fMet) in prokaryotes. The most common start codon is ATG (or AUG in mRNA).

In some embodiments, the inserted sequence further comprises a stop codon (e.g., TAG, TAA, TGA). The stop codon (or termination codon) is a nucleotide triplet within messenger RNA that signals a termination of translation into proteins. Thus, the endogenous sequence after the stop codon will not be translated into proteins. In some embodiments, at least one exon of (e.g., exon 1, exon 2, exon 3, exon 4, exon 5, exon 6, exon 7, exon 8, exon 9, exon 10, and/or exon 11) of the endogenous IL1RAP gene are not translated into proteins.

The genetically modified animal can have one or more cells expressing a human or chimeric IL1RAP (e.g., humanized IL1RAP) having an extracellular region and a cytoplasmic region, wherein the extracellular region comprises a sequence that is at least 50%, 60%, 70%, 80%, 90%, 95%, 99% identical to one of the extracellular regions of human IL1RAP. In some embodiments, the extracellular region of the humanized IL1RAP has a sequence that has at least 10, 20, 30, 40, 50, 60, 70, 80, 90, 100, 110, 120, 130, 140, 150, 160, 170, 180, 200, 220, 240, 260, 280, 300, 320, or 340 amino acids (e.g., contiguously or non-contiguously) that are identical to one of the extracellular regions of human IL1RAP. In some embodiments, the genome of the genetically modified animal comprises a sequence encoding an amino acid sequence that corresponds to part or the entire sequence of exon 1, exon 2, exon 3, exon 4, exon 5, exon 6, exon 7, exon 8, exon 9, exon 10, and/or exon 11 of human IL1RAP, or part or the entire sequence of the extracellular region of human IL1RAP (with or without signal peptide).

In some embodiments, the non-human animal can have, at an endogenous IL1RAP gene locus, a nucleotide sequence encoding a chimeric human/non-human IL1RAP polypeptide, wherein a human portion of the chimeric human/non-human IL1RAP polypeptide comprises a portion of the human IL1RAP extracellular region, and wherein the animal expresses a functional IL1RAP on a surface of a cell of the animal. The human portion of the chimeric human/non-human IL1RAP polypeptide can comprise a portion of exon 1, exon 2, exon 3, exon 4, exon 5, exon 6, exon 7, exon 8, exon 9, exon 10, and/or exon 11 of human IL1RAP. In some embodiments, the human portion of the chimeric human/non-human IL1RAP polypeptide can comprise a sequence that is at least 80%, 85%, 90%, 95%, or 99% identical to SEQ ID NO: 2.

In some embodiments, the non-human animal genome also includes other genetic modifications. In some embodiments, the other genes include one or more of human IL-1 receptor, IL-1, PD-1, PD-L1, CTLA4, LAG3, IL4, IL6, and CCR4 genes. In some embodiments, the other genes include one or more of H2-D, B2M, PD-1, PD-L1, CTLA4, B7H3, B7H4, CD47 and IL23A. In some embodiments, the other genes include one or more of PD-1, PD-L1, IL6, TNF, 41BB, CD40, IL17, TNFR2, IL4, IL33, TIGIT, OX40, IL1B, IL1R1, IL13, CCR5, CCR8 and IL10.

In some embodiments, the nucleotide sequence of the humanized IL1RAP includes one of the following groups:
(1) all or part of the nucleotide sequence shown in SEQ ID NO: 7;
(2) at least 60%, 65%, 70%, 75%, 80%, 85%, 90%, 91%, 92%, 93%, 94%, 95%, 96%, 97%, 98% or at least 99% identity with the nucleotide sequence shown in SEQ ID NO: 7;
(3) no more than 10, 9, 8, 7, 6, 5, 4, 3, 2 or no more than 1 nucleotide difference from the nucleotide sequence shown in SEQ ID NO: 7; or
(4) the nucleotide sequence shown in the nucleotide sequence shown in SEQ ID NO: 7, including substitution, deletion and/or insertion of one or more nucleotides.

In some embodiments, the humanized IL1RAP gene further comprises an auxiliary sequence, which is connected after the human IL1RAP gene. Further preferably, the auxiliary sequence is selected from a stop codon, a flip sequence or a knockout sequence. More preferably, the auxiliary sequence is 3′UTR and/or polyA of a non-human animal.

In some embodiments, the non-human animal can have transcribed mRNA sequence including one of the following groups:
(1) all or part of the nucleotide sequence shown in SEQ ID NO: 7;
(2) at least 60%, 65%, 70%, 75%, 80%, 85%, 90%, 91%, 92%, 93%, 94%, 95%, 96%, 97%, 98% or at least 99% identity with the nucleotide sequence shown in SEQ ID NO: 7;
(3) no more than 10, 9, 8, 7, 6, 5, 4, 3, 2 or no more than 1 nucleotide difference from the nucleotide sequence shown in SEQ ID NO: 7; or
(4) the nucleotide sequence shown in the nucleotide sequence shown in SEQ ID NO: 7, including substitution, deletion and/or insertion of one or more nucleotides.

In some embodiments, the humanized IL1RAP gene also includes a specific inducer or repressor. Further preferably, the specific inducer or repressor can be a conventional inducing or repressing substance.

In a specific embodiment of the present invention, the specific inducer is selected from the tetracycline system (Tet-Off System/Tet-On System) or the tamoxifen system (Tamoxifen System).

In some embodiments, the non-human portion of the chimeric human/non-human IL1RAP polypeptide comprises the transmembrane and/or cytoplasmic region of an endogenous non-human IL1RAP polypeptide. In some embodiments, the non-human portion of the chimeric human/non-human IL1RAP polypeptide comprises the amino acids 368-388, 389-570 or 368-570 of SEQ ID NO: 1.

Furthermore, the genetically modified animal can be heterozygous with respect to the replacement or insertion at the endogenous IL1RAP locus, or homozygous with respect to the replacement or insertion at the endogenous IL1RAP locus.

In some embodiments, the genetically modified animal (e.g., a rodent) comprises a humanization of an endogenous IL1RAP gene, wherein the humanization comprises a replacement at the endogenous rodent IL1RAP locus of a nucleic acid comprising an exon of a IL1RAP gene with a nucleic acid sequence comprising at least one exon of a human IL1RAP gene to form a modified IL1RAP gene.

In some embodiments, the genetically modified animal (e.g., a rodent) comprises an insertion at the endogenous rodent IL1RAP locus of a nucleic acid sequence comprising at least one exon of a human IL1RAP gene to form a modified IL1RAP gene.

In some embodiments, the expression of the modified IL1RAP gene is under control of regulatory elements at the endogenous IL1RAP locus. In some embodiments, the modified IL1RAP gene is operably linked to a WPRE element.

In some embodiments, the humanized IL1RAP locus lacks a human IL1RAP 5′-UTR. In some embodiment, the humanized IL1RAP locus comprises a rodent (e.g., mouse) 5′-UTR. In some embodiments, the humanization comprises a human 3′-UTR. In appropriate cases, it may be reasonable to presume that the mouse and human IL1RAP genes appear to be similarly regulated based on the similarity of their 5′-flanking sequence. As shown in the present disclosure, humanized IL1RAP mice that comprise an insertion at an endogenous mouse IL1RAP locus, which retain mouse regulatory elements but comprise a humanization of IL1RAP encoding sequence, do not exhibit obvious pathologies. Both genetically modified mice that are heterozygous or homozygous for humanized IL1RAP are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized IL1RAP gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized IL1RAP in the genome of the animal.

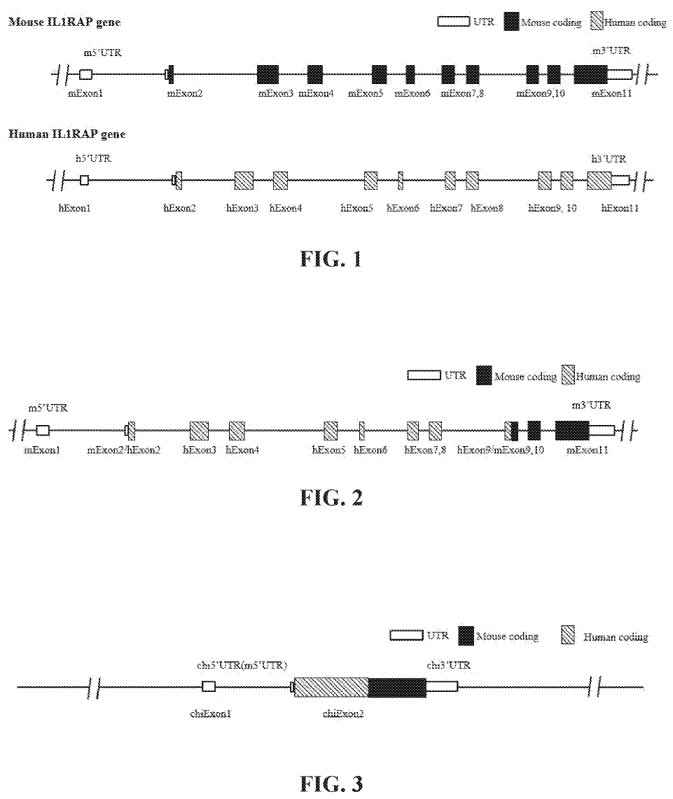

In some embodiments, the non-human mammal comprises the genetic construct as described herein (e.g., gene construct as shown in FIGS. 1-4). In some embodiments, a non-human mammal expressing human or humanized IL1RAP is provided. In some embodiments, the tissue-specific expression of human or humanized IL1RAP protein is provided.

In some embodiments, the expression of human or humanized IL1RAP in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a rodent, e.g., a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human IL1RAP protein or chimeric IL1RAP protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized IL1RAP protein.

In some embodiments, the humanized IL1RAP protein comprises all or part of the amino acid sequence encoded by exons 1 to 11 of the human IL1RAP gene. In some embodiments, the humanized IL1RAP protein comprises all or part of the human IL1RAP amino acid sequence encoded by any one, two, three or more, two consecutive or three or more consecutive exons of the human IL1RAP gene. In some embodiments, the humanized IL1RAP protein comprises the entire amino acid sequence encoded by exons 1-11 of the human IL1RAP gene.

In some embodiments, the humanized IL1RAP protein comprises the amino acid sequence encoded by a part of exon 2, all of exon 3, exon 4, exon 5, exon 6, exon 7, exon 8 and a part of exon 9 of the human IL1RAP gene. In some embodiments, the part of exon 2 of the human IL1RAP gene contains at least the start codon to the last nucleotide of exon 2. In some embodiments, the part of exon 2 contains at least 10 bp (for example, 10, 11, 12, 13, 14, 15, 20, 21, 22, 23, 24, 25, 30, 35, 40, 45, 50, 55, 60, 64 bp). In some embodiments, the part of exon 2 contains at least 20 bp. In some embodiments, the part of exon 2 contains at least 30 bp. In some embodiments, the part of exon 2 contains 64 bp. In some embodiments, the part of exon 9 contains at least from the first nucleotide of exon 9 to the stop codon. In some embodiments, the part of exon 9 contains at least 10 bp (for example, 10, 11, 12, 13, 14, 15, 20, 21, 22, 23, 24, 25, 30, 35, 40, 45, 50 bp). In some embodiments, the part of exon 2 contains at least 20 bp. In some embodiments, the part of exon 2 contains at least 30 bp. In some embodiments, the part of the exon 9 contains 50 bp.

In some embodiments, the signal peptide, the transmembrane region and the cytoplasmic region of the humanized IL1RAP protein are derived from non-human animals. In some embodiments, the extracellular region of at most 30 amino acids is derived from the extracellular region of the non-human animal IL1RAP protein. In some embodiments, the extracellular region of the humanized IL1RAP protein comprises 0-10 amino acids of the extracellular region of non-human animal IL1RAP protein at the N-terminal and/or 0-20 amino acids of the extracellular region of non-human animal IL1RAP protein at the C-terminal.

In some embodiments, the construction method comprises using a signal peptide, a transmembrane region, a cytoplasmic region and/or an extracellular region encoding the human IL1RAP protein. The whole or part of the nucleotide sequence is introduced into the non-human animal IL1RAP locus; preferably, the whole or part of the nucleotide sequence comprising the extracellular region encoding the human IL1RAP protein is introduced into the non-human animal IL1RAP locus; A nucleotide sequence encoding at least 100 amino acid sequences of the extracellular region of human IL1RAP protein is introduced into the non-human animal IL1RAP locus; An amino acid sequence of %, 95% or at least 99% identity or comprising an amino acid sequence identical to positions 21-367 of SEQ ID NO: 2 is introduced into the non-human animal IL1RAP locus. In some embodiments, the humanized IL1RAP protein comprises at least the amino acid sequence encoded by SEQ ID NO: 8, or an amino acid sequence that is at least 70%, 75%, 80%, 85%, 90%, 91%, 92%, 93%, 94%, 95%, 96%, 97%, 98% or at least 99% identical to that encoded by SEQ ID NO: 8.

In some embodiments, the IL1RAP humanized animal comprises all or part of exons 1 to 11 of the human IL1RAP gene. In some embodiments, the IL1RAP humanized animal comprises all or part of any one, two, three or more, two consecutive or three or more consecutive exons of the human IL1RAP gene. In some embodiments, the IL1RAP humanized animal comprises the entire exons 1-11 of the human IL1RAP gene.

In a specific embodiment of the present invention, the humanized IL1RAP gene comprises a part of exon 2, all of exon 3, exon 4, exon 5, exon 6, exon 7, exon 8, and a part of exon 9 of the human IL1RAP gene. In some embodiments, the part of exon 2, all of exon 3, exon 4, exon 5, exon 6, exon 7, exon 8, and the part of exon 9 have at least 70%, 75%, 80%, 85%, 90% or at least 95% sequence identity to all or part of the corresponding exons 1 to 11 of human IL1RAP gene. In some embodiments, the part of exon 2, all of exon 3, exon 4, exon 5, exon 6, exon 7, exon 8, and the part of exon 9 of the human IL1RAP gene are identical to all or part of the corresponding exons 1 to 11 of human IL1RAP gene.

In a specific embodiment of the present invention, the humanized IL1RAP gene comprises at least the nucleotide sequence shown in SEQ ID NO: 3 or SEQ ID NO: 4, or a nucleotide sequence having at least 70%, 75%, 80%, 85%, 90%, 91%, 92%, 93%, 94%, 95%, 96%, 97%, 98% or at least 99% identity to the nucleotide sequence shown in SEQ ID NO: 3 or SEQ ID NO: 4.

In some embodiments, the humanized IL1RAP gene comprises at least SEQ ID NO: 5, or nucleotide sequence that is at least 70%, 75%, 80%, 85%, 90%, 91%, 92%, 93%, 94%, 95%, 96%, 97%, 98% or at least 99% identical to SEQ ID NO: 5.

In some embodiments, the humanized IL1RAP gene comprises at least SEQ ID NO: 6, or nucleotide sequence that is at least 70%, 75%, 80%, 85%, 90%, 91%, 92%, 93%, 94%, 95%, 96%, 97%, 98% or at least 99% identical to SEQ ID NO: 6.

In some embodiments, the humanized IL1RAP gene further includes all of exon 1, part of exon 2, part of exon 9, all of exon 10 and all of exon 11 of the non-human animal IL1RAP gene. In some embodiments, the all of exon 1, part of exon 2, part of exon 9, all of exon 10 and all of exon 11 of the non-human animal IL1RAP gene have at least 70%, 75%, 80%, 85%, 90% or at least 95% identity to the corresponding exon 1, exon 2, exon 9, exon 10 and exon 11 of the mouse IL1RAP gene.

### Vectors

The present disclosure relates to a targeting vector, comprising: a) a DNA fragment homologous to the 5′ end of a region to be altered (5′ arm), which is selected from the IL1RAP gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL1RAP gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000082.7; c) the DNA fragment homologous to the 3′ end of the region to be altered (3′ arm) is selected from the nucleotide sequences that have at least 90% homology to the NCBI accession number NC_000082.7.

In some embodiments, a) the DNA fragment homologous to the 5′ end of a region to be altered (5′ arm) is selected from the nucleotides from the position 26438528 to 26442905 of the NCBI accession number NC_000082.7 (SEQ ID NO: 3); c) the DNA fragment homologous to the 3′ end of the region to be altered (3′ arm) is selected from the nucleotides from the position 26442909 to 26446614 of the NCBI accession number NC_000082.7 (SEQ ID NO: 4).

In some embodiments, the targeting vector comprises a 5′ arm that is a 100-10,000 nucleotide long nucleotide sequence of the non-human animal's IL1RAP gene. In some embodiments, the 5′ arm has at least 90% homology with the NCBI accession number NC_000082.7. In some embodiments, the 5′ arm sequence has at least 90% homology with SEQ ID NO: 3. In some embodiments, the targeting vector comprises a 3′ arm that is a 100-10000 nucleotide long nucleotide sequence of the non-human animal's IL1RAP gene. In some embodiments, the 3′ arm has at least 90% homology with the NCBI accession number NC_000082.7. In some embodiments, the 3′ arm sequence has at least 90% homology with SEQ ID NO: 4.

In some embodiments, the targeting vector further comprises a 5′ arm, which is selected from the 100-10,000 nucleotide long nucleotide sequence of the non-human animal IL1RAP genomic DNA; preferably, the 5′ arm and the NCBI accession number are nucleotides with at least 90% homology with the NCBI accession number NC_000082.7; further preferably, the 5′ arm sequence is shown in SEQ ID NO: 3; and/or, the targeting vector further comprises a 3′ arm, which is selected from 100-10000 nucleotide long nucleotide sequence of the genomic DNA of the non-human animal IL1RAP gene; preferably, the 3′ arm has at least 90% homology with the NCBI accession number NC_000082.7; further preferably, the 3′ arm sequence is shown in SEQ ID NO: 4.

In some embodiments, the length of the selected genomic nucleotide sequence in the targeting vector can be more than about 200 bp, 400 bp, 600 bp, 800 bp or 1000 bp.

In some embodiments, the region to be altered is exon 1, exon 2, exon 3, exon 4, exon 5, exon 6, exon 7, exon 8, exon 9, exon 10, and/or exon 11 of IL1RAP gene.

The targeting vector can further include a selection gene marker.

In some embodiments, the sequence of the 5′ arm is shown in SEQ ID NO: 3; and the sequence of the 3′ arm is shown in SEQ ID NO: 4.

In some embodiments, the sequence is derived from human. For example, the target region in the targeting vector is a part or entirety of the nucleotide sequence of a human IL1RAP or a chimeric IL1RAP. In some embodiments, the nucleotide sequence of the humanized IL1RAP encodes the entire or the part of human IL1RAP protein (SEQ ID NO: 2).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous IL1RAP gene locus, a sequence encoding a region of an endogenous IL1RAP with a sequence encoding a corresponding region of human IL1RAP, a sequencing encoding human IL1RAP, or a sequencing encoding chimeric IL1RAP.

In some embodiments, the disclosure provides inserting in at least one cell of the animal, at an endogenous IL1RAP gene locus, a sequence encoding a human IL1RAP or a chimeric IL1RAP.

In some embodiments, the genetic modification occurs in a germ cell, a somatic cell, a blastocyst, or a fibroblast, etc. The nucleus of a somatic cell or the fibroblast can be inserted into an enucleated oocyte.

In some embodiments, the disclosure provides inserting or replacing all or part of a combination of any one, two, three or more, two consecutive or three or more consecutive exons of exons 1 to 11 of the human IL1RAP gene into the non-human animal IL1RAP locus. In some embodiments, a part of exon 2, all of exon 3, exon 4, exon 5, exon 6, exon 7, exon 8, and a part of exon 9 of the human IL1RAP gene is inserted or replaced into a non-human animal IL1RAP locus. In some embodiments, the part of exon 2 of the human IL1RAP gene contains at least the start codon to the last nucleotide of exon 2. In some embodiments, the part of exon 2 contains at least 10 bp (for example, 10, 11, 12, 13, 14, 15, 20, 21, 22, 23, 24, 25, 30, 35, 40, 45, 50, 55, 60, 64 bp) nucleotides. In some embodiments, the part of exon 2 contains 64 bp nucleotides. In some embodiments, the part of exon 9 contains at least from the first nucleotide of exon 9 to the stop codon. In some embodiments, the part of exon 9 contains at least 10 bp (for example, 10, 11, 12, 13, 14, 15, 20, 21, 22, 23, 24, 25, 30, 35, 40, 45, 50 bp) nucleotides. In some embodiments, the part of the exon 9 contains 50 bp nucleotides. In some embodiments, the inserted sequence includes from the start codon to the stop codon of the human IL1RAP gene. In some embodiments, the inserted sequence includes a coding region (CDS) of the human IL1RAP gene.

In some embodiments, the method comprises using a signal peptide, a transmembrane region, a cytoplasmic region and/or an extracellular region encoding the human IL1RAP protein. In some embodiments, the whole or part of the human IL1RAP nucleotide sequence is introduced into the non-human animal's IL1RAP locus. In some embodiments, the whole or part of the nucleotide sequence encoding the extracellular region of the human IL1RAP protein is introduced into the non-human animal's IL1RAP locus. In some embodiments, a nucleotide sequence encoding at least 100 amino acids of the extracellular region of human IL1RAP protein is introduced into the non-human animal's IL1RAP locus. In some embodiments, a nucleotide sequence encoding an amino acid sequence that is 90%, 95% or at least 99% identical to positions 21-367 of SEQ ID NO: 2 is introduced into the non-human animal IL1RAP locus. In some embodiments, the humanized IL1RAP protein comprises the amino acid sequence encoded by SEQ ID NO: 8, or an amino acid sequence that is at least 70%, 75%, 80%, 85%, 90%, 91%, 92%, 93%, 94%, 95%, 96%, 97%, 98% or at least 99% identical to that encoded by SEQ ID NO: 8.

In a specific embodiment of the present invention, the construction method comprises inserting or replacing the cDNA sequence of human IL1RAP into the non-human animal locus. In some embodiments, the construction method comprises inserting or replacing the nucleotide sequence comprising SEQ ID NO: 5 into the non-human animal's endogenous IL1RAP gene locus.

In some embodiments, the construction method comprises replacing all or part of exons 2 to 9 of the non-human animal IL1RAP nucleotide sequence with a cDNA sequence comprising human IL1RAP gene sequence. In some embodiments, the construction method comprises using a cDNA sequence comprising SEQ ID NO: 5 into the non-human animal's endogenous IL1RAP gene locus to replace a part of exon 2, all of exon 3, exon 4, exon 5, exon 6, exon 7, exon 8 and a part of exon 9 of the endogenous non-human animal IL1RAP nucleotide sequence. In some embodiments, the replaced sequence includes introns 2-8 of a non-human animal's endogenous IL1RAP gene. In some embodiments, the part of exon 2 of the non-human animal's endogenous IL1RAP gene includes at least the start codon of the non-human animal IL1RAP gene to the last nucleotide of exon 2.

FIG. 2 shows an exemplary humanization strategy for a mouse IL1RAP locus. In FIG. 2, the targeting strategy involves a vector comprising the 5′ end homologous arm, human IL1RAP gene fragment or chimeric IL1RAP gene fragment, 3′ homologous arm. The process can involve inserting a human sequence by homologous recombination. In some embodiments, the cleavage at the upstream and the downstream of the target site (e.g., by zinc finger nucleases, TALEN or CRISPR) can result in DNA double strand breaks, and the homologous recombination is used to insert the human IL1RAP sequence. In some embodiments, a sequence encoding the human or chimeric IL1RAP gene is used to replace a part of exon 2, all of exon 3, exon 4, exon 5, exon 6, exon 7, exon 8 and a part of exon 9 of the endogenous mouse IL1RAP gene. In some embodiments, the sequence encoding the human or chimeric IL1RAP gene comprises a part of exon 2, all of exon 3, exon 4, exon 5, exon 6, exon 7, exon 8, and a part of exon 9 of the human IL1RAP gene. In some embodiments, the sequence encoding the human or chimeric IL1RAP gene further comprises a polyA sequence. In some embodiments, the sequence encoding the human or chimeric IL1RAP gene further comprises a Neo cassette. In some embodiments, the IL1RAP humanized animal comprises all of exon 1, a part of exon 2, a part of exon 9, all of exon 10, and all of exon 11 of the endogenous IL1RAP gene. In some embodiments, the sequence of the 5′ homology arm is shown in SEQ ID NO: 3 and the sequence of the 3′ homology arm is shown in SEQ ID NO: 4. In some embodiments, the donor sequence is shown in SEQ ID NO: 5.

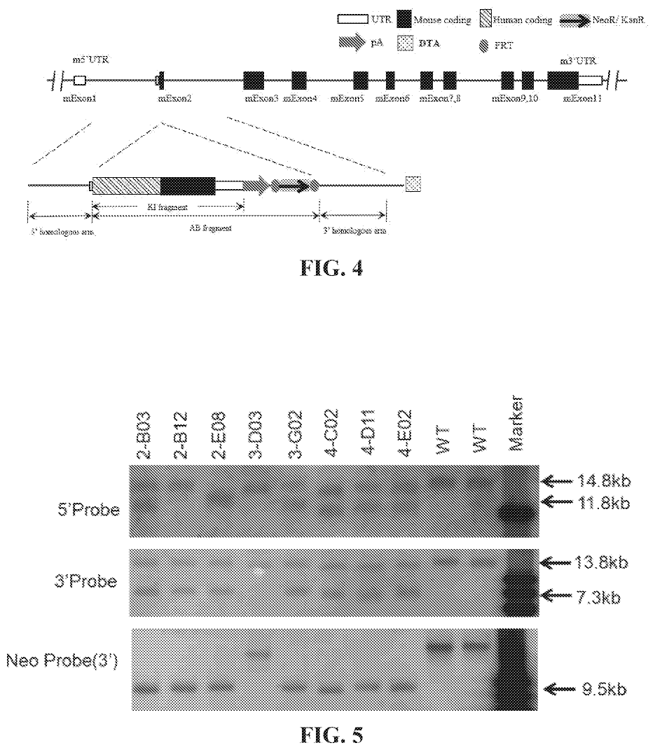

FIG. 4 shows an exemplary humanization strategy for a mouse IL1RAP locus. In FIG. 4, the targeting strategy involves a vector comprising the 5′ end homologous arm, human IL1RAP gene fragment or chimeric IL1RAP gene fragment, 3′ homologous arm. The process can involve inserting a human sequence by homologous recombination. In some embodiments, the cleavage at the upstream and the downstream of the target site (e.g., by zinc finger nucleases, TALEN or CRISPR) can result in DNA double strand breaks, and the homologous recombination is used to insert the human IL1RAP sequence. In some embodiments, a sequence encoding the human or chimeric IL1RAP gene is used to replace a part of the endogenous mouse IL1RAP gene (e.g., one or more nucleotides in exon 2). In some embodiments, the sequence encoding the human or chimeric IL1RAP gene comprises a part of exon 2, all of exon 3, exon 4, exon 5, exon 6, exon 7, exon 8, and a part of exon 9 of the human IL1RAP gene. In some embodiments, the IL1RAP humanized animal comprises all of exon 1, a part of exon 2, exon 3, exon 4, exon 5, exon 6, exon 7, exon 8, exon 9, exon 10 and exon 11 of the endogenous IL1RAP gene. In some embodiments, the sequence of the 5′ homology arm is shown in SEQ ID NO: 3 and the sequence of the 3′ homology arm is shown in SEQ ID NO: 4. In some embodiments, the inserted human sequence is shown in SEQ ID NO: 5. In some embodiments, the inserted mouse sequence is shown in SEQ ID NO: 6.

Thus, in some embodiments, the methods for making a genetically modified, humanized animal, can include the step of inserting at an endogenous IL1RAP locus (or site), a sequence encoding a human IL1RAP or a chimeric IL1RAP. The sequence can include a region (e.g., a part or the entire region) of exon 1, exon 2, exon 3, exon 4, exon 5, exon 6, exon 7, exon 8, exon 9, exon 10, exon 11 of a human IL1RAP gene. In some embodiments, the sequence includes a region of exon 1, exon 2, exon 3, exon 4, exon 5, exon 6, exon 7, exon 8, exon 9, exon 10, exon 11 of a human IL1RAP gene (e.g., SEQ ID NO: 2). In some embodiments, the endogenous IL1RAP locus is exon 1, exon 2, exon 3, exon 4, exon 5, exon 6, exon 7, exon 8, exon 9, exon 10, and/or exon 11 of mouse IL1RAP (e.g., SEQ ID NO: 1).

In some embodiments, the inserted chimeric IL1RAP gene fragment includes a inserted human sequence and a inserted mouse sequence. In some embodiments, the inserted human sequence is identical to the nucleotide sequence from nucleotide 137 to 1237 of NM_001167929.2. In some embodiments, the inserted mouse sequence is identical to the nucleotide sequence from nucleotide 1285 to 1896 of NM_008364.2.

In some embodiments, the methods of modifying a IL1RAP locus of a mouse to express a chimeric human/mouse IL1RAP peptide can include the steps of replacing at the endogenous mouse IL1RAP locus a nucleotide sequence encoding a mouse IL1RAP with a nucleotide sequence encoding a human IL1RAP, thereby generating a sequence encoding a chimeric human/mouse IL1RAP.

The present disclosure further provides a method for establishing a IL1RAP gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, the method for making the genetically modified animal comprises:
(1) providing a plasmid comprising a human IL1RAP gene fragment, flanked by a 5′ homology arm and a 3′ homology arm, wherein the 5′ and 3′ homology arms target an endogenous IL1RAP gene;
(2) providing two small guide RNAs (sgRNAs) that target the endogenous IL1RAP gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized IL1RAP protein; and
(5) mating the child mouse obtained in step (2) to obtain a homozygote mouse,

In some embodiments, the fertilized egg is modified by CRISPR with sgRNAs that target a 5′-terminal targeting site and a 3′-terminal targeting site.

In some embodiments, the humanized IL1RAP protein comprises SEQ ID NO: 8.

In some embodiments, the sequence encoding the humanized IL1RAP protein is operably linked to an endogenous regulatory element at the endogenous IL1RAP gene locus.

In some embodiments, the genetically-modified animal does not express an endogenous IL1RAP protein.

### Methods of Using Genetically Modified Animals

Insertion of human genes in a non-human animal at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized IL1RAP protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized IL1RAP, which are useful for testing agents that can decrease or block the interaction between IL1RAP and IL1RAP ligands (e.g., IL-1 receptor) or the interaction between IL1RAP and anti-human IL1RAP antibodies, testing whether an agent can increase or decrease the IL1RAP pathway activity, and/or determining whether an agent is an IL1RAP agonist or antagonist. The genetically modified animals can be, e.g., an animal model of a human disease, e.g., the disease is induced genetically (a knock-in or knockout). In various embodiments, the genetically modified non-human animals further comprise an impaired immune system. In various embodiments, the non-human animal is genetically modified to sustain or maintain a human xenograft, such as a human solid tumor or a blood cell tumor (e.g., a lymphocyte tumor, e.g., a B or T cell tumor).

In some embodiments, the genetically modified animals can be used for determining effectiveness of a IL1RAP targeting agent for the treatment of autoimmune diseases. The methods involve administering the agent (e.g., anti-human IL1RAP antibody) to the animal as described herein, wherein the animal has an autoimmune disease; and determining the efficacy. In some embodiments, the agent is an anti-human IL1RAP antibody.

In some embodiments, the anti-human IL1RAP antibody binds to one or more of the four isoforms of IL1RAP (i.e., mIL-IRAcP, sIL-IRAcP, sIL-IRAcP-β and AcPb). In some embodiments, the anti-human IL1RAP antibody binds to mIL-IRAcP. In some embodiments, the anti-human IL1RAP antibody binds to sIL-IRAcP. In some embodiments, the anti-human IL1RAP antibody binds to sIL-1RAcP-β. In some embodiments, the anti-human IL1RAP antibody binds to AcPb.

In some embodiments, the genetically modified animals can be used for determining whether an agent (e.g., an anti-IL1RAP antibody or a fusion protein) is a IL1RAP agonist or antagonist. In some embodiments, the methods as described herein are also designed to determine the effects of the agent (e.g., anti-IL1RAP antibodies) on IL1RAP, e.g., whether the agent can change the IL1RAP mediated signal transduction of the genetically modified animals. In some embodiments, the genetically modified animals can be used for determining the effective dosage of a therapeutic agent for treating a disease in the subject, e.g., immune disorders.

In some embodiments, the agent is designed for treating various immune-related diseases. Thus, the methods as described herein can be used to determine the effectiveness of an agent targeting IL1RAP (e.g., anti-IL1RAP antibody) in treating the immune-related diseases. The immune-related diseases include but are not limited to allergies, asthma, dermatitis, myocarditis, nephritis, hepatitis, systemic lupus erythematosus, rheumatoid arthritis, scleroderma, hyperthyroidism, and primary thrombocytopenia Purpura, autoimmune hemolytic anemia, ulcerative colitis, autoimmune liver disease, diabetes, pain or neurological disorders, etc.

“Autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include neoplastic cells.

Accordingly, in some embodiments, the autoimmune diseases to be evaluated using the genetically modified animals described herein, include, but are not limited to: rheumatoid arthritis, Crohn's disease, ulcerative colitis, multiple sclerosis, primary sclerosing cholangitis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenia purpura, scleroderma with anti-collagen antibodies, mixed connective tissue disease, polymyositis, pernicious anemia, idiopathic Addison's disease, autoimmune-associated infertility, Kawasaki's disease, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Autoimmune disease has been recognized also to encompass atherosclerosis and Alzheimer's disease. In one embodiment of the aspects described herein, the autoimmune disease is selected from the group consisting of multiple sclerosis, type-I diabetes, Hashimoto's thyroiditis, Crohn's disease, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis and myasthenia gravis.

In some embodiments, the genetically modified animals can be used for determining effectiveness of a drug for the treatment of cancer. In some embodiments, the methods involve administering a drug to the animal as described herein, wherein the animal has a tumor; and determining the inhibitory effects of the drug to the tumor.

The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the tumor comprises one or more cancer cells (e.g., human or mouse cancer cells) that are injected into the animal. In some embodiments, the drug is an IL1RAP antibody.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the drug is an IL1RAP antibody. In some embodiments, the dosage of the drug is equal to or more than 1 mg/kg, 2 mg/kg, 5 mg/kg, 10 mg/kg, 15 mg/kg, 20 mg/kg, 25 mg/kg, 30 mg/kg, 40 mg/kg or 50 mg/kg. In some embodiments, the dosage of the drug is equal to or less than 1 mg/kg, 2 mg/kg, 5 mg/kg, 10 mg/kg, 15 mg/kg, 20 mg/kg, 25 mg/kg, 30 mg/kg, 40 mg/kg or 50 mg/kg. In some embodiments, the dosage of the drug is 1-5 mg/kg, 1-10 mg/kg, 1-20 mg/kg, 1-30 mg/kg, 1-50 mg/kg, 2-5 mg/kg, 2-10 mg/kg, 2-20 mg/kg, 2-30 mg/kg, 2-50 mg/kg, 5-10 mg/kg, 5-20 mg/kg, 5-30 mg/kg, 5-50 mg/kg, 10-20 mg/kg, 10-30 mg/kg or 10-50 mg/kg.

In some embodiments, the drug can reduce the tumor volume in the IL1RAP humanized animal as compared with the IL1RAP humanized animal treated with control. In some embodiments, the TGI_{TV} % in the treatment group is more than 5%, 7.5%, 10%, 12.5%, 15%, 17.5%, 20%, 22.5%, 25%, 27.5%, 30%, 35%, 40%, 45%, 50%, 55%, or 60%. In some embodiments, the TGI_{TV} % in the treatment group is less than 5%, 7.5%, 10%, 12.5%, 15%, 17.5%, 20%, 22.5%, 25%, 27.5%, 30%, 35%, 40%, 45%, 50%, 55%, or 60%. In some embodiments, the TGI_{TV} % in the treatment group is 5%-10%, 5%-20%, 5%-30%, 5%-40%, 5%-50%, 5%-60%, 10%-20%, 10%-30%, 10%-40%, 10%-50%, 10%-60%, 20%-30%, 20%-40%, 20%-50%, 20%-60%, 30%-40%, 30%-50% or 30%-60%.

In some embodiments, the p value of body weight change in the treatment group is equal to or more than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1. In some embodiments, the p value of body weight change in the treatment group is equal to or more than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1. In some embodiments, the p value of body weight change in the treatment group is 0.01-0.1, 0.01-0.2, 0.01-0.5, 0.01-1, 0.05-0.1, 0.05-0.2, 0.05-0.5, 0.05-1, 0.1-0.2, 0.1-0.5, or 0.1-1.

In some embodiments, the p value of tumor volume change in the treatment group is equal to or more than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1. In some embodiments, the p value of tumor volume change in the treatment group is equal to or more than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1. In some embodiments, the p value of tumor volume change in the treatment group is 0.01-0.1, 0.01-0.2, 0.01-0.5, 0.01-1, 0.05-0.1, 0.05-0.2, 0.05-0.5, 0.05-1, 0.1-0.2, 0.1-0.5, or 0.1-1.

In some embodiments, the drug is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the drug is designed for treating chronic myeloid leukemia (CML), acute myeloid leukemia (AML), myelodysplastic syndrome or solid tumor (e.g., non-small cell lung cancer (NSCLC), pancreatic cancer, breast cancer, colorectal cancer, liver cancer, esophageal cancer, head and neck cancer, bladder cancer). In some embodiments, the drug is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the drug is designed for treating hepatocellular, ovarian, colon, or cervical carcinomas. In some embodiments, the drug is designed for treating advanced breast cancer, advanced ovarian cancer, and/or advanced refractory solid tumor. In some embodiments, the drug is designed for treating metastatic solid tumors, NSCLC, melanoma, non-Hodgkin lymphoma, colorectal cancer, and multiple myeloma. In some embodiments, the drug is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the drug is designed for treating carcinomas (e.g., nasopharynx carcinoma, bladder carcinoma, cervix carcinoma, kidney carcinoma or ovary carcinoma).

In some embodiments, the present disclosure provides a tumor-bearing or inflammation model in the evaluation of a treatment of immune-related diseases, tumors and/or inflammation. The present disclosure also provides a method for screening a specific modulator. The screening method includes applying the modulator to an individual implanted with tumor cells to detect tumor suppressive properties; wherein, the individual is selected from the aforementioned non-human animal, the non-human animal obtained by the aforementioned construction method, or the aforementioned tumor-bearing or metabolic disease model. In some embodiments, the modulator is a modulator targeting IL1RAP.

In some embodiments, the modulator is selected from CAR-T and drugs. Further preferably, the drug is an antibody or a small molecule drug.

In some embodiments, the modulator is a monoclonal antibody or a bispecific antibody or a combination of two or more drugs.

In some embodiments, the detection includes determining the size and/or proliferation rate of tumor cells.

In some embodiments, the detection method includes vernier caliper measurement, flow cytometry detection and/or in vivo animal imaging detection.

In some embodiments, the detection includes assessing individual body weight, fat mass, activation pathway, neuroprotective activity, or metabolic changes, and the metabolic changes include changes in food consumption or water consumption.

In some embodiments, the tumor cells are derived from human or non-human animals.

In some embodiments, the screening method for the modulator is not a treatment method. This screening method is used to screen or evaluate drugs, test and compare the efficacy of candidate drugs to determine which candidate drugs can be used as drugs and which cannot be used as drugs, or to compare the sensitivity of different drugs, that is, the therapeutic effect is not inevitable and is just a possibility.

The present disclosure also provides methods of determining toxicity of a drug (e.g., a drug that targets IL1RAP). The methods involve administering the drug to the animal as described herein. The animal is then evaluated for its weight change, red blood cell count, hematocrit, and/or hemoglobin. In some embodiments, the drug can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the IL1RAP gene function, human IL1RAP antibodies, drugs for human IL1RAP targeting sites, the drugs or efficacies for human IL1RAP targeting sites, the drugs for metabolic disorders, the drugs for immune-related diseases and antitumor drugs.

Genetically Modified Animal Model with Two or More Human or Chimeric Genes

The present disclosure further relates to methods for generating genetically modified animal model with two or more human or chimeric genes. The animal can comprise a human or chimeric IL1RAP gene and a sequence encoding an additional human or chimeric protein.

In some embodiments, the additional human or chimeric protein can be IL-1, IL-1 receptor, cytotoxic T-lymphocyte-associated protein 4 (CTLA-4), Lymphocyte Activating 3 (LAG-3), B And T Lymphocyte Associated (BTLA), Glucagon-like peptide-1 (IgG), CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), TNF Receptor Superfamily Member 4 (TNFRSF4 or OX40), programmed cell death protein 1 (PD-1), or programmed cell death 1 ligand (PD-L1).

The methods of generating genetically modified animal model with two or more human or chimeric genes (e.g., humanized genes) can include the following steps:
(a) using the methods of introducing human IL1RAP gene or chimeric IL1RAP gene as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric IL-1, IL-1 receptor, PD-1, CTLA-4, LAG-3, BTLA, CD27, CD28, CD40, CD47, CD137, CD154, TIGIT, TIM-3, GITR, SIRPa, or OX40. Some of these genetically modified non-human animals are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the IL1RAP humanization is directly performed on a genetically modified animal having a human or chimeric IL-1, IL-1 receptor, CTLA-4, BTLA, CD27, CD28, CD40, CD47, CD137, CD154, TIGIT, TIM-3, GITR, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., a drug targeting IL1RAP and an additional therapeutic agent for the treatment of various disease. The methods include administering the drug targeting IL1RAP and the additional therapeutic agent to the animal, wherein the animal has a tumor; and determining the inhibitory effects of the combined treatment to the tumor. In some embodiments, the additional therapeutic agent is an antibody that specifically binds to IL-1, IL-1 receptor, CTLA-4, BTLA, CD27, CD28, CD40, CD47, CD137, CD154, TIGIT, TIM-3, GITR, or OX40. In some embodiments, the additional therapeutic agent is an anti-CTLA4 antibody (e.g., ipilimumab), an anti-PD-1 antibody, or an anti-CTLA4 antibody.

### EXAMPLES

The invention is further described in the following examples, which do not limit the scope of the invention described in the claims.

In the following examples, equipment and materials were obtained from the companies indicated below:
DraIII and EcoRV enzymes were purchased from NEB, and the product numbers are R3510 and R3195S respectively;
C57BL/6 mice and Flp tool mice were purchased from the National Rodent Laboratory Animal Seed Center, National Institutes for Food and Drug Control;
Brilliant Violet 510™ anti-mouse CD45 was purchased from Biolegend, Cat. No. 103138;
Purified anti-mouse CD16/32 Antibody was purchased from Biolegend, Cat. No. 101302;
Zombie NIR™ Fixable Viability Kit from Biolegend, Catalog No. 423106;
PerCP anti-mouse Ly-6G/Ly-6C (Gr-1) Antibody was purchased from Biolegend, Cat. No. 108426;
V450 Rat Anti-mouse CD11b was purchased from BD Biosciences, Cat. No. 560455;
FITC anti-mouse F4/80 Antibody was purchased from Biolegend, Cat. No. 123108;
Human IL-1 RAcP/IL-1 R3 PE-conjugated Antibody was purchased from R&D, Cat. No. FAB676P.

### Example 1. Preparation of IL1RAP Gene Humanized Mice

Mouse IL1RAP gene (NCBI Gene ID: 16180, Primary source: MGI: 104975, UniProt: Q61730, located at positions 26400259 to 26548878 of chromosome 16 NC_000082.7, based on transcript NM_008364.2 and its encoded protein NP_032390.1 (SEQ ID NO: 1)) and human IL1RAP gene (NCBI Gene ID: 3556, Primary source: HGNC: 5995, UniProt ID: Q9NPH3, located at positions 190514085 to 190659750 of chromosome 3 NC_000003.12, based on transcript NM_001167929.2 and its encoded protein NP_001161401.1 (SEQ ID NO: 2)) are used. A schematic diagram of the comparison between mouse IL1RAP gene and human IL1RAP gene is shown in FIG. 1.

To prepare IL1RAP gene humanized mice, a nucleotide sequence encoding human IL1RAP protein can be introduced into the mouse endogenous IL1RAP locus, so that the mouse expresses a human or humanized IL1RAP protein. For example, using gene editing techniques, sequences encoding the human signal peptide and extracellular region are knocked into the mouse locus under the control of the regulatory element of the mouse IL1RAP gene. In one embodiment, as shown in FIG. 2, exon 2 to exon 9 of human IL1RAP gene is used to replace the corresponding part of the mouse IL1RAP gene. Alternatively, as shown in FIG. 3, the fragment containing a human nucleotide sequence or a human-mouse hybrid sequence is inserted into the specific site of the mouse endogenous, and the insertion is made so that expression of the endogenous sequence after the insertion site is terminated.

Following the strategy in FIG. 3, the targeting strategy shown in FIG. 4 is designed. The targeting vector contains the homology arm sequences upstream and downstream of the mouse IL1RAP gene, and the knock-in AB fragment, where the AB fragment comprises the KI fragment (SEQ ID NO: 35) containing the nucleotide sequence of human IL1RAP. Among them, the upstream homology arm sequence (5′ homology arm, SEQ ID NO: 3) is the same as the nucleotide sequence from nucleotide 26438528 to 26442905 of NCBI accession number NC_000082.7, and the downstream homology arm sequence (3′ homology arm, SEQ ID NO: 4) is the same as the nucleotide sequence from nucleotide 26442909 to 26446614 of the NCBI accession number NC_000082.7; the knock-in fragment contains the coding sequence of human IL1RAP and the coding sequence of murine IL1RAP, where the coding sequence of human IL1RAP (SEQ ID NO: 5) is identical to the nucleotide sequence from nucleotide 137 to 1237 of NCBI accession number NM_001167929.2, and the murine IL1RAP coding sequence (SEQ ID NO: 6) is identical to the nucleotide sequence from nucleotide 1285 to 1896 of NCBI accession number NM_008364.2. The mRNA sequence of the humanized IL1RAP gene is shown in SEQ ID NO: 7, and the expressed amino acid sequence thereof is shown in SEQ ID NO: 8.

There are also auxiliary sequences on the targeting vector located downstream of the murine coding sequence, including the 3′UTR, polyA (pA) (SEQ ID NO: 9), and a resistance gene for positive clone selection-namely the coding sequence of neomycin phosphotransferase (Neo), and two site-specific Frt recombination sites arranged in the same direction on both sides of the resistance gene, forming a Neo cassette. The 5′ end of the Neo cassette is directly connected to pA, and the junction sequence between the 3′ end of the Neo cassette and the mouse sequence is designed as 5′-TTCATCAGTCAGGTACATAATTAGGTGGATCCACTAGTTCTAGAGCGGCCGCGGACTT CTGTGGTATTTGATGAGTCTGTCCTTCTATGGGATCCTGC-3′ (SEQ ID NO: 10), where the last “C” of the sequence “CCGC” “is the last nucleotide of the Neo cassette, and the first “G” of the sequence “GGACT” is the first nucleotide of the mouse sequence. In addition, a negative selection marker (the encoding gene for the A subunit of diphtheria toxin (DTA)) was included downstream of the 3′ homology arm of the targeting vector.

Given that human IL1RAP and murine IL1RAP have multiple isoforms or transcripts, the methods described herein can be applied to other isoforms or transcripts.

The construction of the targeting vector can be made, e.g., by enzyme cleavage and ligation. The constructed targeting vector is preliminarily verified by enzyme digestion, and then sent to a sequencing company for sequencing verification. The target vector verified by sequencing was electroporated into embryonic stem cells of wild-type mice, and the obtained cells were screened using the positive screening marker. PCR (primers shown in Table 3) and Southern Blot technology were used to screen the obtained cells to verify the integration of foreign sequences. The clones identified as positive by PCR were then verified by Southern Blot (cell DNA was digested with DraIII or EcoRV and hybridized with 3 probes; the probe sizes are shown in Table 4). The results are shown in FIG. 5. The detection results indicate that among the positive clones, 5 clones (2-B03, 3-G02, 4-C02, 4-D11, 4-E02) were positive heterozygous clones without random insertion.

TABLE 3
PCR primers and target fragment sizes

| | | Fragment |
|---|---|---|
| Primer | Sequence (5′-3′) | Size |
| IL1RAP-F1 | AGGGAGGATTGTGAGGCAATA | 4772 bp |
| | CCAG (SEQ ID NO: 11) | |
| IL1RAP-R1 | GTTCAAAGAGTGGGCACTTGA | |
| | TGCG (SEQ ID NO: 12) | |
| IL1RAP-F2 | GCTCGACTAGAGCTTGCGGA | 4227 bp |
| | (SEQ ID NO: 13) | |
| IL1RAP-R2 | TGGAGCCAAGACGTGTTACGT | |
| | TAGC (SEQ ID NO: 14) | |

Southern Blot detection includes the following probe primers:

5′Probe:
F:
(SEQ ID NO: 15)
5′-TCATGGTTGTCTGTGAGCCAGAGGA-3′
R:
(SEQ ID NO: 16)
5′-AGAGAACGCAAATGCAGAAAGCAGG-3′
3′Probe:
F:
(SEQ ID NO: 17)
5′-GTACACATGTGCCACAGGGAAGGT-3′
R:
(SEQ ID NO: 18)
5′-CTCTGGGTCCTGGTGGCACTCTATC-3′
Neo Probe:
F:
(SEQ ID NO: 19)
5′-GGATCGGCCATTGAACAAGATGG-3′
R:
(SEQ ID NO: 20)
5′-CAGAAGAACTCGTCAAGAAGGCG-3′

TABLE 4
Table 3: Southern blot probes and target fragment sizes

| Restriction | | wild-type | Recombinant |
|---|---|---|---|
| endonuclease | Probe | fragment size | fragment size |
| DraIII | 5′Probe | 14.8 kb | 11.8 kb |
| EcoRV | 3′Probe | 13.8 kb | 7.3 kb |
| DraIII | Neo Probe (3′) | — | 9.5 kb |

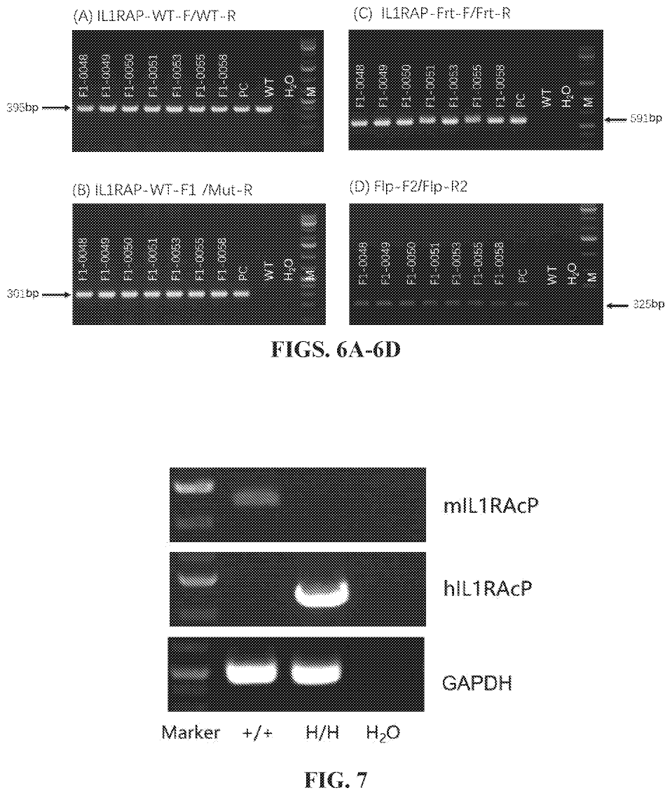

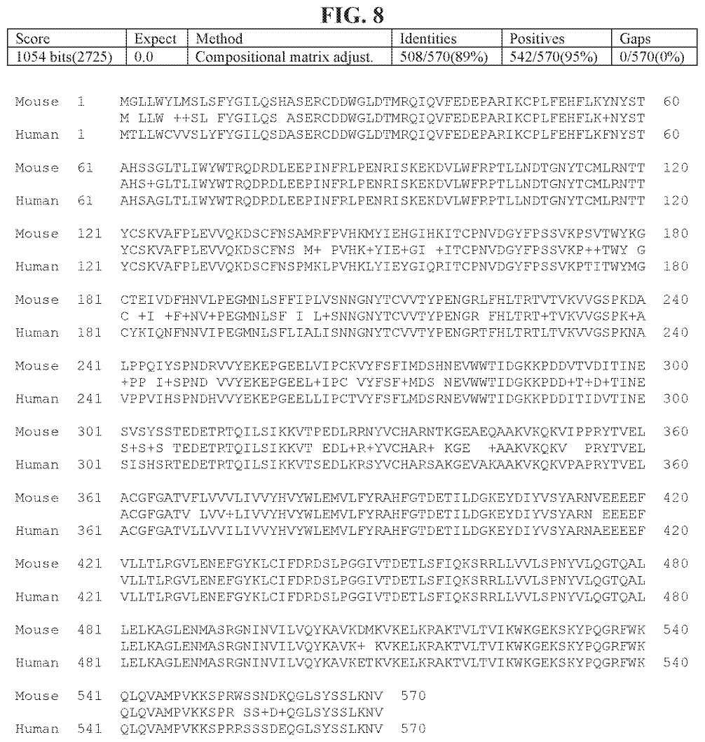

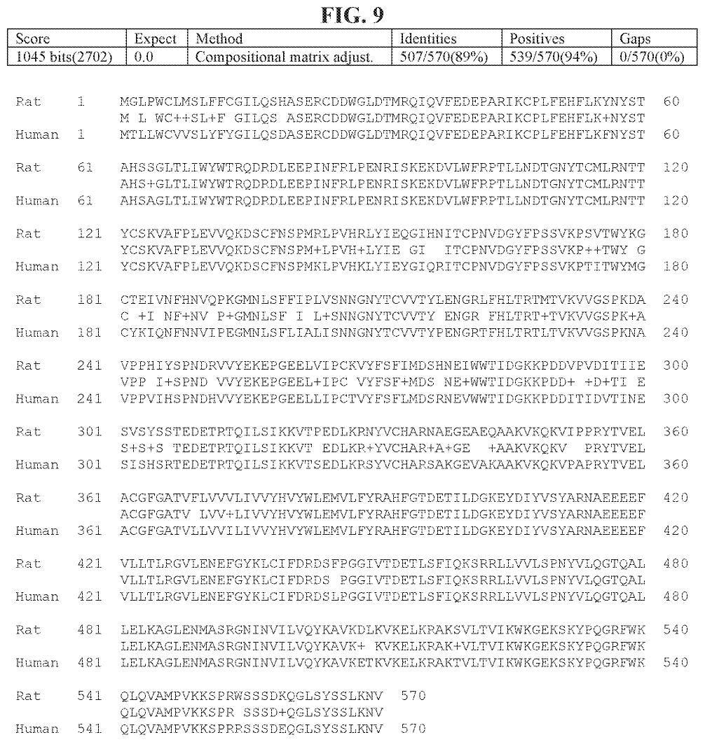

The screened correct positive cloned cells (black mice) were introduced into the isolated blastocysts (white mice), and the obtained chimeric blastocysts were transferred to a cell culture medium for a short-term culture and then transplanted to the oviduct of recipient female mice (white mice) to produce F0 generation chimeric mice (black and white). The F0 generation chimeric mice and wild-type mice were backcrossed to obtain the F1 generation mice, and then the F1 generation heterozygous mice were mated to one another to obtain the F2 generation homozygous mice. Also, the Flp tool mouse can be mated with the positive mice to remove the screening marker gene, and then the IL1RAP gene humanized homozygous mouse can be obtained by mating with each other. The genotype of the offspring mouse somatic cells can be determined by PCR (primers are shown in Table 5), and the results of several F1 generation mice (neo marker being removed) are shown in FIG. 6, wherein F1-0048, F1-0049, F1-0050, F1-0051, F1-0053, F1-0055 and F1-0058 mice were heterozygous mice with the humanized gene.

TABLE 5
PCR primers and target fragment sizes

| | | Fragment |
|---|---|---|
| Primer | Sequence (5′-3′) | size |
| IL1RAP-WT-F | TGTTGGTTCTGACAATAAAGC | WT: |
| | TTGGG (SEQ ID NO: 21) | 395 bp |
| IL1RAP-WT-R | CAGACCCCTTCCTAAGTTGTC | |
| | TAGT (SEQ ID NO: 22) | |
| IL1RAP-WT-F1 | GTTGAGACTCAGAAAGACGGG | Mut: |
| | CAAT (SEQ ID NO: 23) | 301 bp |
| IL1RAP-Mut-R | GTTCAAAGAGTGGGCACTTGA | |
| | TGCG (SEQ ID NO: 24) | |
| IL1RAP-Frt-F | AGGATCACTCCACTTGTTAAC | Mut: |
| | TACCC (SEQ ID NO: 25) | 591 bp |
| IL1RAP-Frt-R | ACATACTCACCCGAAGCATGA | (removing |
| | CTCT (SEQ ID NO: 26) | neo) |
| Flp-F2 | GACAAGCGTTAGTAGGCACAT | Mut: |
| | ATAC (SEQ ID NO: 27) | 325 bp |
| Flp-R2 | GCTCCAATTTCCCACAACATT | |
| | AGT (SEQ ID NO: 28) | |

WT is the wild-type target band, and Mut is the target band for successful IL1RAP recombination.

RT-PCR can be used to detect the expression of humanized IL1RAP mRNA in IL1RAP humanized mice. One 12-week-old wild-type C57BL/6 mouse and one 12-week-old IL1RAP humanized homozygous mouse were selected, and the liver tissues were collected after euthanasia. The total cellular RNA was extracted, which was reverse transcribed into cDNA using a reverse transcription kit for PCR amplification. Primer sequences are shown in Table 6 below.

TABLE 6
RT-PCR primers and target fragment sizes

| Primer | | Target |
|---|---|---|
| and SEQ ID | Sequence (5′-3′) | Fragment |
| mIL1RAcP-F1 | CTTCCGCCTCCCAGAG | WT: |
| (SEQ ID NO: 29) | AATC | 1178 bp |
| mIL1RAcP-R1 | TTCCCTGGAGCACGTA | |
| (SEQ ID NO: 30) | GTTG | |
| hIL1RAcP-F1 | CCTGCAAAGTGATGCC | Mut: |
| (SEQ ID NO: 31) | TCAGAA | 1228 bp |
| hIL1RAcP-R1 | CGTCAGCAGCACAAAT | |
| (SEQ ID NO: 32) | TCCTC | |
| GAPDH-F | TCACCATCTTCCAGGA | WT: |
| (SEQ ID NO: 33) | GCGAGA | 479 |
| GAPDH-R | GAAGGCCATGCCAGTG | |
| (SEQ ID NO: 34) | AGCTT | |

The test results (FIG. 7) showed that in wild-type C57BL/6 mouse cells, murine IL1RAP mRNA was detected, and humanized IL1RAP mRNA was not detected. By contrast, in humanized IL1RAP homozygous mouse cells, humanize IL1RAP mRNA was detected, and murine IL1RAP mRNA was not detected.

The in vivo expression of humanized IL1RAP protein in mice was confirmed by flow cytometry. 7-8 week old wild-type C57BL/6 mice and IL1RAP humanized homozygous mice were selected, and spleen cells were collected. Cells were labeled with Zombie NIR (BioLegend), human IL1RAP/IL-1R3 PE-conjugated Antibody (hIL1RAcP-PE), Brilliant Violet 510™ anti-mouse CD45, PerCP anti-mouse Ly-6G/Ly-6C (Gr-1) Antibody, V450 Rat anti-mouse CD11b, FITC anti-mouse F4/80 Antibody. The results of flow cytometry showed that, compared with wild-type C57BL/6 mice cells, in in the spleen of humanized mice, anti-human IL1RAP antibody could detect monocytes (referred to as Mon, labeled as CD45+Gr1−CD11b+F4/80−) and macrophages (referred as Mo, labeled as CD45+Gr1−CD11b+F4/80+) that express humanized IL1RAP protein on the cell surface. The proportion of hIL1RAP positive cells in wild-type mouse macrophages was 0.86%, and the proportion of hIL1RAP positive cells in IL1RAP humanized homozygous macrophages was 21.8%. The proportion of hILRAcP in wild-type mouse monocytes was 0.99%, and the proportion of hILRAcP in IL1RAP gene humanized homozygous monocytes was 9.23%. This indicated that the humanized IL1RAP protein could be successfully expressed in the spleen of IL1RAP humanized homozygous mice.

### Example 2. Preparation of Double Humanized or Multiple Humanized Mice

The method of Example 1 can be modified to prepare double or multiple humanized mouse models. For example, in the aforementioned Example 1, the embryonic stem cells used for blastocyst microinjection can be selected from mice containing IL33, PD-L1, IL10, IL1B, IL1R1, IL13, IL17, CCR5, CCR8, and other genetic modifications. The prepared IL1RAP gene humanized mouse model can also be used to prepare double or multiple humanized mouse models. For example, on the basis of the IL1RAP humanized mice, IL1RAP and other gene-modified double-gene or multi-gene-modified mouse models can be obtained using the isolation of mouse embryonic stem cells and gene recombination targeting technology. The homozygous or heterozygous IL1RAP humanized mouse obtained by this method can also be mated with other gene-modified homozygous or heterozygous mice, and the offspring are screened. According to Mendelian inheritance, there is a certain probability to obtain heterozygous mice containing both a humanized IL1RAP and one or more additional modified genes. The heterozygotes can be mated with each other to obtain double gene or multigene modified homozygotes. These double gene or multigene modified mice can be used to test the efficacy of human IL1RAP targeting drugs and drugs targeting other genes.

### Example 3. Testing Efficacy Using the IL1RAP Humanized Mouse Model

The IL1RAP humanized mice prepared by this method can be used to evaluate the efficacy of drugs targeting human IL1RAP. For example, IL1RAP humanized homozygous mice are subcutaneously inoculated with mouse colon cancer cells MC38, and when the tumor volume reach about 100 mm³, the mice were slotted into a control group or a treatment group according to the tumor volume. The treatment group mice are treated with drugs targeting human IL1RAP. The control group mice are treated with an equal volume of normal saline or PBS. The tumor volume and body weight of the mice are determined periodically, and the in vivo safety and efficacy of the drug can be effectively assessed by comparing the changes in the body weight of the mice and the tumor volume.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric interleukin-1 receptor accessory protein (IL1RAP).

2. The animal of claim 1, wherein the sequence encoding the human or chimeric IL1RAP is operably linked to an endogenous regulatory element at the endogenous IL1RAP gene locus in the at least one chromosome.

3. The animal of claim 1 or 2, wherein the sequence encoding the human or chimeric IL1RAP comprises a sequence encoding an amino acid sequence that is at least 70%, 75%, 80%, 85%, 90%, 95%, or 99% identical to SEQ ID NO: 8.

4. The animal of any one of claims 1-3, wherein the sequence encoding the human or chimeric IL1RAP comprises a sequence encoding an amino acid sequence that is at least 70%, 75%, 80%, 85%, 90%, 95%, or 99% identical to amino acids 1-367 of SEQ ID NO: 2 or amino acids 21-367 of SEQ ID NO: 2.

5. The animal of any one of claims 1-4, wherein the animal is a mammal, e.g., a monkey, a rodent or a mouse.

6. The animal of any one of claims 1-5, wherein the animal is a mouse or a rat.

7. The animal of any one of claims 1-6, wherein the animal does not express endogenous IL1RAP or expresses a decreased level of endogenous IL1RAP as compared to that of an animal without genetic modification.

8. The animal of any one of claims 1-7, wherein the animal has one or more cells expressing human or chimeric IL1RAP.

9. The animal of any one of claims 1-8, wherein the sequence encoding the human or chimeric IL1RAP comprises a part of exon 2, exon 3, exon 4, exon 5, exon 6, exon 7, exon 8 and a part of exon 9 of the human IL1RAP nucleotide sequence.

10. The animal of claim 9, wherein the part of exon 2 comprises at least 10 bp of the human IL1RAP nucleotide sequence, and the part of exon 9 comprises at least 10 bp of the human IL1RAP nucleotide sequence.

11. The animal of any one of claims 1-10, wherein the human or chimeric IL1RAP protein comprises an amino acid sequence that is identical to SEQ ID NO: 8.

12. The animal of any one of claims 1-11, wherein the sequence encoding the human or chimeric IL1RAP comprises a nucleotide sequence that is at least 70%, 75%, 80%, 85%, 90%, 91%, 92%, 93%, 94%, 95%, 96% %, 97%, 98% or at least 99% identical to the nucleotide sequence shown in SEQ ID NO: 3, SEQ ID NO: 4, SEQ ID NO: 5, SEQ ID NO: 6, SEQ ID NO: 7, or SEQ ID NO: 10.

13. The animal of any one of claim 1-12, wherein the human or chimeric IL1RAP gene further comprises exon 1, a part of exon 2, a part of exon 9, exon 10 and exon 11 of the non-human animal's endogenous IL1RAP gene.

14. A genetically-modified, non-human animal, wherein the genome of the animal comprises a replacement of a sequence encoding a region of endogenous IL1RAP with a sequence encoding a corresponding region of human IL1RAP at an endogenous IL1RAP gene locus.

15. The animal of claim 14, wherein the sequence encoding the corresponding region of human IL1RAP is operably linked to or under the control of an endogenous regulatory element (e.g., 5′ UTR or 3′ UTR) at the endogenous IL1RAP locus.

16. The animal of claim 14 or 15, wherein the animal does not express endogenous IL1RAP or expresses a decreased level of endogenous IL1RAP as compared to that of an animal without genetic modification.

17. The animal of any one of claims 14-16, wherein one or more cells of the animal expresses a chimeric IL1RAP.

18. The animal of any one of claims 14-17, wherein the animal has one or more cells expressing a chimeric IL1RAP having a humanized extracellular region, transmembrane region, and/or cytoplasmic region, wherein the humanized extracellular region comprises a sequence that is at least 50%, 60%, 70%, 80%, 90%, 95%, or 99% identical to the corresponding extracellular region of human IL1RAP.

19. The animal of any one of claims 14-18, wherein the human or chimeric IL1RAP comprises a sequence that is at least 70%, 75%, 80%, 85%, 90%, 91%, 92%, 93%, 94%, 95%, 96%, 97%, 98% or at least 99% identical to SEQ ID NO: 8.

20. The animal of any one of claims 14-19, wherein the genome of the animal comprises at least SEQ ID NO: 3, SEQ ID NO: 4, SEQ ID NO: 5, SEQ ID NO: 6, SEQ ID NO: 7, SEQ ID NO: 10, or a nucleotide sequence that is at least 70%, 75%, 80%, 85%, 90%, 91%, 92%, 93%, 94%, 95%, 96%, 97%, 98% or at least 99% identical to the nucleotide sequence set forth in SEQ ID NO: 3, SEQ ID NO: 4, SEQ ID NO: 5, SEQ ID NO: 6, SEQ ID NO: 7, or SEQ ID NO: 10.

21. The animal of any one of claims 14-20, wherein the animal further comprising a deletion of one or more nucleotide from the endogenous IL1RAP gene.

22. The animal of any one of claims 14-21, wherein the animal further comprises an endogenous IL1RAP gene 5′-UTR, and/or an endogenous IL1RAP gene 3′-UTR.

23. The animal of any one of claims 14-22, wherein the animal is heterozygous or homozygous with respect to the replacement at the endogenous IL1RAP gene locus.

24. A genetically-modified, non-human animal, wherein the genome of the animal comprises an insertion of a sequence encoding a human or a chimeric IL1RAP at an endogenous IL1RAP gene locus.

25. The animal of claim 24, wherein the inserted sequence is operably linked to an endogenous regulatory element at the endogenous IL1RAP locus, and one or more cells of the animal express human IL1RAP or chimeric IL1RAP.

26. The animal of claim 24 or 25, wherein the animal does not express endogenous IL1RAP or expresses a reduced amount of endogenous IL1RAP.

27. The animal of any one of claims 24-26, wherein the inserted sequence is located immediately after 5′-UTR at the endogenous IL1RAP locus.

28. The animal of any one of claims 24-27, wherein the animal has one or more cells expressing a chimeric IL1RAP having one or more humanized extracellular regions, transmembrane regions, and cytoplasmic regions, wherein one or more of the humanized extracellular regions comprise a sequence that is at least 50%, 60%, 70%, 80%, 90%, 95%, or 99% identical to the corresponding extracellular region of human IL1RAP.

29. The animal of any one of claims 24-28, wherein one or more of the humanized extracellular regions of the chimeric IL1RAP has a sequence that has at least 10, 20, 30, 40, 50, 60, 70, 80, 90, 100, 200, 220, 240, 260, 280, 300, 320 or 340 contiguous amino acids that are identical to a contiguous sequence present in the corresponding extracellular region of human IL1RAP.

30. The animal of any one of claims 24-29, further comprising a deletion of one or more nucleotide from the endogenous IL1RAP gene (e.g., at exon 2).

31. The animal of any one of claims 24-30, wherein the animal is heterozygous with respect to the insertion at the endogenous IL1RAP gene locus.

32. The animal of any one of claims 24-30, wherein the animal is homozygous with respect to the insertion at the endogenous IL1RAP gene locus.

33. A method for making a genetically-modified non-human animal, the method comprising: replacing in at least one cell of the animal, at an endogenous IL1RAP gene locus, a sequence encoding a region of an endogenous IL1RAP with a sequence encoding a corresponding region of human IL1RAP.

34. The method of claim 33, wherein the sequence encoding the corresponding region of human IL1RAP gene comprises exon 1, exon 2, exon 3, exon 4, exon 5, exon 6, exon 7, exon 8, exon 9, exon 10, and/or exon 11, or a part thereof, of a human IL1RAP gene.

35. The method of claim 33 or 34, wherein the sequence encoding the corresponding region of human IL1RAP gene encodes a sequence that is at least 90% identical to amino acids 1-367 of SEQ ID NO: 2 or amino acids 21-367 of SEQ ID NO:2.

36. The method of any one of claims 33-35, wherein the sequence encoding the corresponding region of human IL1RAP gene is at least 90% identical to SEQ ID NO: 5.

37. The method of any one of claims 33-36, wherein the sequence encoding a region of human IL1RAP gene is identical to SEQ ID NO: 5.

38. The method of any one of claims 33-37, wherein the method further comprises deleting one or more nucleotides of the endogenous IL1RAP gene.

39. The method of any one of claims 33-38, wherein a part of exon 2, all of exon 3, exon 4, exon 5, exon 6, exon 7, exon 8, and a part of exon 9 of the endogenous IL1RAP are replaced.

40. The method of claim 39, wherein the sequence encoding a region of the endogenous IL1RAP is replaced by a part of exon 2, all of exon 3, exon 4, exon 5, exon 6, exon 7, exon 8, and a part of exon 9 of the human IL1RAP gene.

41. The method of claim 40, wherein the part of exon 2 of the human IL1RAP gene comprises at least 10 bp contiguous human nucleotides, wherein the part of exon 9 of the human IL1RAP gene comprises at least 10 bp contiguous human nucleotides.

42. A method for making a genetically-modified, non-human animal, comprising:
inserting in at least one cell of the animal, at an endogenous IL1RAP gene locus, a sequence encoding a region of human IL1RAP gene.

43. The method of claim 42, wherein the sequence encoding the region of human IL1RAP gene comprises a part of exon 2, exon 3, exon 4, exon 5, exon 6, exon 7, exon 8, and a part of exon 9 of a human IL1RAP gene.

44. The method of claim 42 or 43, wherein the sequence encoding a region of human IL1RAP gene encodes a sequence that is at least 90% identical to SEQ ID NO: 2.

45. The method of any one of claims 42-44, wherein the animal is a mouse, and the endogenous IL1RAP locus is within exon 2 of the mouse IL1RAP gene.

46. The method of any one of claims 42-45, further comprising deleting one or more nucleotides of the endogenous IL1RAP gene.

47. A non-human animal comprising at least one cell comprising a nucleotide sequence encoding a humanized IL1RAP polypeptide, wherein the humanized IL1RAP polypeptide comprises at least 50 contiguous amino acid residues that are identical to the corresponding contiguous amino acid sequence of a human IL1RAP, wherein the animal expresses the humanized IL1RAP.

48. The animal of claim 47, wherein the humanized IL1RAP polypeptide has at least 50 contiguous amino acid residues that are identical to the corresponding contiguous amino acid sequence of a human IL1RAP extracellular region.

49. The animal of claim 47 or 48, wherein the humanized IL1RAP polypeptide comprises a sequence that is at least 90%, 95%, or 99% identical to amino acids 1-367 of SEQ ID NO: 2 or amino acids 21-367 of SEQ ID NO: 2.

50. The animal of any one of claims 47-49, wherein the nucleotide sequence is operably linked to an endogenous IL1RAP regulatory element of the animal (e.g., 5′-UTR).

51. The animal of any one of claims 47-50, wherein the humanized IL1RAP polypeptide comprises a humanized extracellular region, an endogenous IL1RAP transmembrane region and/or an endogenous IL1RAP cytoplasmic region.

52. The animal of any one of claims 47-51, wherein the nucleotide sequence is integrated to an endogenous IL1RAP gene locus of the animal.

53. A method of making a genetically-modified mouse cell that expresses a chimeric IL1RAP, the method comprising:
replacing at an endogenous mouse IL1RAP gene locus, a nucleotide sequence encoding a region of mouse IL1RAP with a nucleotide sequence encoding a corresponding region of human IL1RAP, thereby generating a genetically-modified mouse cell that includes a nucleotide sequence that encodes the chimeric IL1RAP, wherein the mouse cell expresses the chimeric IL1RAP.

54. The method of claim 53, wherein the chimeric IL1RAP polypeptide comprises a sequence that is at least 90%, 95%, or 99% identical to amino acids 1-367 of SEQ ID NO: 2 or amino acids 21-367 of SEQ ID NO: 2.

55. The method of claim 53, wherein the chimeric IL1RAP comprises:
the extracellular region of human or humanized IL1RAP; and
the transmembrane region and/or the cytoplasmic region of mouse IL1RAP.

56. The animal of any one of claims 1-32 and 47-52, wherein the animal further comprises a sequence encoding an additional human or chimeric protein.

57. The animal of claim 56, wherein the additional human or chimeric protein is IL-1, IL-1 receptor, PD-1, PD-L1, IL6, TNF, 41BB, CD40, IL17, TNFR2, IL4, IL33, TIGIT, OX40, IL1B, IL1R1, IL13, CCR5, CCR8 or IL10.

58. The method of any one of claims 33-46 and 53-55, wherein the animal or mouse further comprises a sequence encoding an additional human or chimeric protein.

59. The method of claim 58, wherein the additional human or chimeric protein is IL-1, IL-1 receptor, PD-1, PD-L1, IL6, TNF, 41BB, CD40, IL17, TNFR2, IL4, IL33, TIGIT, OX40, IL1B, IL1R1, IL13, CCR5, CCR8 or IL10.

60. A method of determining effectiveness of a therapeutic agent targeting IL1RAP for the treatment of an immune-related disease, the method comprising:
administering the therapeutic agent targeting IL1RAP to the animal of any one of claims 1-32 and 47-52, wherein the animal has an immune-related disease; and
determining the effects of the therapeutic agent targeting IL1RAP to the immune-related disease of the animal.

61. The method of claim 60, wherein the immune-related disease is an autoimmune disease.

62. The method of claim 60, wherein determining the effects of the therapeutic agent targeting IL1RAP to the immune-related disease of the animal comprises measuring the T cell proliferation, cytokine secretion and killing capacity.

63. The method of claim 60, wherein determining the effects of the therapeutic agent targeting IL1RAP to the immune-related disease of the animal further comprises assessing individual body weight, fat mass, activation pathway, neuroprotective activity, or metabolic changes, and the metabolic changes include changes in food consumption or water consumption.

64. A method of determining effectiveness of a human or humanized antibody for the treatment of a disease, the method comprising
administering the antibody to the animal of any one of claims 1-32 and 47-52; and
determining the effects of the antibody on the disease.

65. The method of claim 64, wherein the disease is a tumor.

66. The method of claim 64, wherein the animal further comprises a sequence encoding a human or chimeric IL-1, IL-1 receptor, PD-1, PD-L1, IL6, TNF, 41BB, CD40, IL17, TNFR2, IL4, IL33, TIGIT, OX40, ILIB, IL1R1, IL13, CCR5, CCR8 or IL10.

67. The method of claim 64, wherein the antibody is an anti-IL1RAP antibody.

68. A method of determining effectiveness of an anti-IL1RAP antibody for the treatment of cancer, the method comprising:
administering the anti-IL1RAP antibody to the animal of any one of claims 1-32 and 47-52, wherein the animal has a tumor; and
determining the inhibitory effects of the anti-IL1RAP antibody to the tumor.

69. The method of claim 68, wherein the tumor comprises one or more cancer cells that are injected into the animal.

70. The method of claim 68, wherein determining the inhibitory effects of the anti-IL1RAP antibody to the tumor comprises measuring the tumor volume in the animal.

71. The method of claim 68, wherein the tumor cells are derived from non-small cell lung cancer (NSCLC), pancreatic cancer, breast cancer, colorectal cancer, liver cancer, esophageal cancer, head and neck cancer, bladder cancer, or colon cancer.

72. A protein comprising an amino acid sequence, wherein the amino acid sequence is one of the following:
(a) an amino acid sequence set forth in SEQ ID NO: 8;
(b) an amino acid sequence that is at least 90% identical to SEQ ID NO: 8;
(c) an amino acid sequence that is at least 91%, 92%, 93%, 94%, 95%, 96%, 97%, 98%, or 99% identical to SEQ ID NO: 8;
(d) an amino acid sequence that is different from the amino acid sequence set forth in SEQ ID NO: 8 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 8.

73. A nucleic acid comprising a nucleotide sequence, wherein the nucleotide sequence is one of the following:
(a) a sequence that encodes the protein of claim 58;
(b) SEQ ID NO: 5;
(c) SEQ ID NO: 7;
(d) SEQ ID NO: 10;
(e) a sequence that is at least 90% identical to SEQ ID NO: 5, SEQ ID NO: 7, SEQ ID NO: 10;
(f) a sequence that is at least 91%, 92%, 93%, 94%, 95%, 96%, 97%, 98%, or 99% identical to SEQ ID NO: 5, or SEQ ID NO: 7, SEQ ID NO: 10.

74. A cell comprising the protein of claim 58 and/or the nucleic acid of claim 59.

75. An animal comprising the protein of claim 58 and/or the nucleic acid of claim 59.

76. A method of determining toxicity of an anti-IL1RAP antibody, the method comprising administering the anti-IL1RAP antibody to the animal of any one of claims 1-32 and 47-52; and determining weight change of the animal.

77. The method of claim 76, the method further comprising performing a blood test (e.g., determining red blood cell count).


SEQUENCE LISTING (36 sequences)

SEQ ID NO: 1 - protein, 570 aa, Mus musculus
MGLLWYLMSLSFYGILQSHASERCDDWGLDTMRQIQVFEDEPARIKCPLFEHFLKYNYSTAHSSGLTLIWYWTRQDRDLEEPINFRLPENRISKEKDVLWFRPTLLNDTGNYTCMLRNTTYCSKVAFPLEVVQKDSCFNSAMRFPVHKMYIEHGIHKITCPNVDGYFPSSVKPSVTWYKGCTEIVDFHNVLPEGMNLSFFIPLVSNNGNYTCVVTYPENGRLFHLTRTVTVKVVGSPKDALPPQIYSPNDRVVYEKEPGEELVIPCKVYFSFIMDSHNEVWWTIDGKKPDDVTVDITINESVSYSSTEDETRTQILSIKKVTPEDLRRNYVCHARNTKGEAEQAAKVKQKVIPPRYTVELACGFGATVFLVVVLIVVYHVYWLEMVLFYRAHFGTDETILDGKEYDIYVSYARNVEEEEFVLLTLRGVLENEFGYKLCIFDRDSLPGGIVTDETLSFIQKSRRLLVVLSPNYVLQGTQALLELKAGLENMASRGNINVILVQYKAVKDMKVKELKRAKTVLTVIKWKGEKSKYPQGRFWKQLQVAMPVKKSPRWSSNDKQGLSYSSLKNV

SEQ ID NO: 2 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 3 - DNA, 4378 bp, Artificial Sequence
  5′ homology arm
gtgtgttaatggaaaccatattccagcttatctgtaatttagagaatagtaaggaaaaccacagatgggcttccaatagctgaggactcagagaagcagtttggtagcagtattgatctgacaaagctttggccatctatggcttgtgctctgctaggggccaagtgttccatggttagatcattacaaagaacataaacagtaagtaaaatcagatccatggccgcaggaggcttatcaaacttgtagggtgactttgtaggatacagacacagtaaaattcccaggaagaaaacctgacaactgctcattcttaaggtctgagtctagcgattctggtgcatcataggagcgacaattcttcaagcataggagaatacatagagtcagctaacagataaccgctaccctgcggctcagggaaatgccattgtgcaaacaaaatctgagcaactggctttgaggccttgagcctagaatgctggtccagggagggaacacctgaatgctaactcaaagcctaggccaaccagaaggttatcgagcttcgagaatgtggataatggcaagtttttgtataaaggtaggtgtaagagtcaggaggagaaagtgaagccttactaacatttctatgcctatgtagaattaagcatgcatttgaaactcctgaacctaaggatatgataggaaatagaatggataacaggaaagactggctgggcacatatgtagatctctataatctgaagctaggacgggatgcactagacaccatcacagttatataaaactagctgcaattcaggtactgtaagtcagtttttctatctcctactaaatagcctctcattccaagatccaataattgaaagtgtattcctttttacaatgacagaaattccaaggctgagtgcaagtcagtattaaatgaaccatcatcctctaagagggttcaggctagccagccatctcccactaacattggtacctttagtccgtcagaatatcctgcacaatccaatttcagttttgtcggcttgatgttttattcatcacagggcaaggttgaccctggatttgtatttgaacaaaggttatctttggattagctctctgttggtgacctgactctgctccatcgtgcataatccagtaactcttgggaatatcaattaaggtcctcaatcacactaattagaatgtcttctatgtgttgccctaggaactcagcaacttataaacaggtaattggtgaaattcaaagagaggtatgtgtgtgcagatctgtgtattagcctccctcccacccctatctgtgtgtgtgtgtgtgtgtgtgtgtgtgtgtgtgtgtgtgtgtaaatgcaatgaggaaggtcagaatactttagtaaggctggaactgctgcgctctgaatctgcagtgcaaaatcttcttgatcttactgacaaggattgatgtggaattattaattacttttattaatgacttgggtgaggacataggtggcatgctgagcaagtgtgtcaatgacaaaagcctagcaggagagcagtaactgtgatgacaaattcaggatccaaaattactccaagacagattttagtggccattctcttttgttatgtgcctggcagagtgaccttcctaagtcctaagttgataactcgtgtgttactgtatcaggtctctacccagaaaacagttatttagtctgatatgaaagttaactcacttgggggccctgtgctccatccaatagctgactgtgagcatctacttatgtgtttgctaggccccggcctagtctcacaagagacagctatatcagggccctttcagcaaaatcttgctagtgtatgcaatggtgtcctcttttggaggctaattatgggatggatctctggatatggcagtctctagatggtccatccttttgtctcagctccaaagtttgtctctgtaactccttccatggaggaactagagaaagtatccaaggagctaaagagatctgcaaccctgtaggtacaacaacattatgaactaaccagtaccccggagctcttgactctagctgcatatgtatcaaaagatggcctagtcggctatcactggaaagagagacccattggacatgcaaactttatatgccccaatacaggggaacaccagggccaaaaaatgggaatgggtgggtagggaagtgtgggggagggtatgggggacttttgggatagcattctaaatgtaattgaggaaaatacgtaataataaaaaatattaaaaaaaaaaaagaaagttaactcacatgtctttggcttgcttttctcggcaccggcaccttctcttacaggccctttctgctttacaattgaatgatctgcggctctcttcccaataccctgtacttgagccaaacagtagtatcactagatggttattacactatttttattatgtctcttattttctttatctttgaatgttagcctccaagactctcatttctccggatgctccttctgcttcaattcccaataaacaacatgttctccctcagtgtgaaagccttgtgttccagcttcccggaggctttggttggagcaatctctcatttgttcatcttgagttgtattggcttgttgctttcttttaccacattttactttgtactccagtttgttcttttggtacatatttgacgtttccataagactgcttactactatgttctctgtctgatccgtcatatttcttgtctgtgtctgcacatatatattttatgcatagttgaagacattgaggtagtcttcatggctttactgactgtagagaaatattcatcaaaggcctttcattatgcttactagaatccactgaagttagatgaatgtaacttattaataaaaataagtaaaatactctggggagaaagatggtgctggggctcaagacgtgggtgttgcatgtcttacggaagtgctgtcccaacagggtacctctgaagctctcaacatgtttttattgaatgagtgaatgaatgatagatggatggatacaactaaattctcctaagataatggtctgaggactggaatgtagctgtccaaggaaaggagatttgatatttgaaacaaaatgttttcatgcaacacctcttgtgataaacttatctgtgatttttatgtagataacctaaggaaggaattagagatatttgatggtctgttgttaagataaatggtaggtgtagaaattatatacaatacagatgaagctgtttgggtaataccaatttatacaattcatttccatgtagtactcttataaatagcccattttggtgtattatgtcaagctatcaagtatttgttttggccattttaggtatgtgggttcctgtgcttgatgtttttgttgttgctattgttgttttttgtttattttttgtaatatcccatctatgttgctttctaaaagtatcatgtgcacaagctaccagatgtccctaagcatgtctgtatgaatgctttcatcaattagaaggcagtcatgagccaggaatttctgagtcattccatatttctgtatttataaccctggagatattttaccattgagaggcatataaaaaggtgtactttacagagttgttaaatctcatgcactaaagaccatacaaccaagtataggagcagggcccttgctagttggcagtttcctctggttatcgtcagaatgctaaaattcacagacatttctgccttgccatttgtctttgcagtgtataaacaagagcggcagggcagaaggtttccatatttcttgatgatataaattctgtgtggttgactatagactaggtttggatccaggatctctagatgttcaattcattactgagacgaaacagtacctggatcataacatatttcccactgagactacacaagggacctgaaagaagggtgagagggtcaagttcatctgttagaagtttcactgcaggtcagaaaatccagatgaactttttttttttccatatagcagttatagttcagtaattagcagagaattctgcagcctcacagctggaatgtaatgatggccagaccactttttagtcaagggatctcagttatgttatttaaactttttttgagtttatctctctgtaatttggggatttaaaagcaaatgatgataaacagcaaaagctatcaaatttgttgttggttctgacaataaagcttgggaatattctaaaacccttggaatggttcttagtctatagttatcacacattcacggtgattattattagaactcttcagggaagaagtaccctctcttactttttaaatttatgttgacagg

SEQ ID NO: 4 - DNA, 3706 bp, Artificial Sequence
  3′ homology arm
ggacttctgtggtatttgatgagtctgtccttctatgggatcctgcagagtcatgcttcgggtgagtatgtggtgctggataaatgactgaaattaatttaacatacaggatcctatatagtgaatttaaatgagcataatgttggttttgttccacagaattctgtagttgcatagaatcaagtgtctgaaagaaccatagaattaggggatgagagaaactagacaacttaggaaggggtctgatgagtataaacacagggacatctcttcaacttcatgccccacaggctaattctctgtattctcatctcaagtactgtggaggaaatggcagggagattatacaacctggctctcggactttgaccactgggatgttgaagaaagaaacaaacaaaccaaaaatgatagttttgatcaaactgcatgtatataggcaacaaagaatttgttggtatgctccttctttgaccatggctttgtatgaggagtcaaccaagatatttcttgagtttgaataaccaaacgtagtcaccctgatggtggatcacatgtaagccagaggtaggaaaagttggctatatgtctctcatctctaaattgcttttcagtgtcacccatgtttgctctatctagttagcatctaagtgcaggttgttatatttttttttgactcattgagtttttataaatattcttgcccatgggactgaaattagtctctgccatttccatatatttctttgatttagtttcatgattgaaaaagaaaatacctaaagggctgcttcttgaatttttacgcagggactccagggatcctaattttaataaggatcctgatgtaaaattcctgtgcacacgatgggctctgctttgactgtcattgctttaatgcctattctctgtgagaaccctttcgaagtgttaagtcagcattatgtaactgtgaccccagtgttctgccatccgtaccttcaactcctcttcccttggcatgaatttcagccctcctgccatcctgatcatctttgctgggtgcatttcagtttttctatgccacttagagcattgtattagaaaccaaaaataactctaaagacagggtgtaactaggacagagtagagctgaaagtacagataatatctaaacttgctggcccattgggacttcacagaggagaggctgtattctaaccaggccttctatccaactgggatgcagtaatacagctgggccacattaatataccagcatctaggcggtatctgtctcgtttgattggaagggacttgctgtactgattcttaaatatgctgacgataaccctggactataatttattttcacttgacttattactaattgaatgttattttcttacagaggctttccttgaccattttaattgcaatagccctgctagtcactcctctaccctcttccattgctttaacatttcccatcatctgccaatattccattaactctttatggtacttcttctctgtcatttgtccttcaatccatccttatcctcttctccactgtctttttcttctttccaacctctcttctttccttgtctcctacttctttacctgtcatctccctctctctgaatctgatttcacagatttatttatcttcctttttgttttcccaaacacttgaaatgatgattaacacatagagatgcagtacaagtgcaaaaatgataaacagatgggtaaatatcccatccgaggcaccttttctcaactggacccattcgtgttgctaacatttaattccacactcatgattcccattagcatgacaagtaccatgcctggaagttaaagtgagagatgtccatgctactgctgttgagagatctaggcttcccagagtttgggaggtctattgcctgcccaatggtcctgacagtgccgcctttctgaatgaccttcactgtattctcccagagatccccttcccccaaatgctgttggtaagtgcactttcttcccaggaactgtgactcataattctgtcaggggaagctgccttcttgaataagatgagttctcataccagctccattgagtcacgatgggagaggaaacatgttgtaatcccctctgcccatctggcacacactcatgccttccttatgaagaggtgtattgtgtctgtgtctctgctgtacttcctcattggagggaggggaacaaaaaggaagtgttgttgacaaaaaagaaaaaaatgaaaggggcaactggctctggggataaaagggatcattgcgatcttgaaggatctgccaatctctctataggatggtgtcacctctgagagggaccgagatgtaggcagacttgttggtcttccgtgaggctctctgtggttcagtttcacttcttcgtgaaaatacacactaacataaggaactgaggagaaatatcccgagtgatctaccgtactctatattcataggatacaagtcagggatttggcattctggacccagagttgactgtcatacaatgaggttcctgtgcagagaattggctttcaaggcctaaaactgtgacaccagagtgtggtcttctggatggaaaatttgctttcctcatacctttaaaaatttggaaggaaacatttcatctcaaataataataatttaaagaataaacagcaacaacaacaacaacaacaacaacaatataataacaagtataacttgccattaaggaattaaagcttggataattttgttagtgacaacataatgaaatgtggctcttttagaagtcatgcaactccttttccttaaatgctttctgaactacccagatcatgactattgtaactctgtctttttcactagacacttacatcctgtacagttcaatgagttatatccacacagctagtattcagcataactgaagtttccacttgtttttcacttccaaaaatgcctgctatttttattaaatatggatcccctgggctccaccaaaagtgccaatgatgtaaagagaaggttgtgatgcaaagagcagcagagataccattagggaagtccctccctcactctggaagaactgcctggtcaggattctatgctgtctacttaagcggttacatctttaccattccctcaagaccattttcttcacatggaagattgctgcaattctcatttcaaacctcagtgctcccccttttgcttgttatacttctcagcatttccactattagctggaaggagttcctgcttacaggaattaaaagggaaaaatatcagttcagatcatccctccccattgatcccttttcccattgtctacctgttagagggctggactgctagagctacctctgagtaaaaatacatatttggtgacctctttggctcagagttggagagacagtgaaatgagtgtccaggaggataaacttgaaaatcttgcagtatcaaaaatttgctttctctgtagatcctattttttttaaaataggcaatgcatttattgactccccaaaatgtgtgggcacaaagatctatggatgtgctttgttgttgttgttgttgtttcaaagtcaacactgataaatatggaacatgcatagggttagattttgcacacatgtcccaggaggagggagtccttgcaatttaggaaatcaaggacttagaagttggctgagctg

SEQ ID NO: 5 - DNA, 1101 bp, Artificial Sequence
  nucleotide sequence from nucleotide 137 to 1237 of NCBI accession number NM_001167929.2
atgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaactggcttgtggttttggagccaca

SEQ ID NO: 6 - DNA, 612 bp, Artificial Sequence
  nucleotide sequence from nucleotide 1285 to 1896 of NCBI accession number NM_008364.2
gtctttctggtagtggttctcattgtggtttaccatgtttactggctggagatggtcctcttttaccgagctcactttggaacagatgaaacaattcttgatggaaaggagtatgatatttatgtttcctatgcaagaaatgtggaagaagaggaatttgtgctgctgacgctgcgtggagttttggagaatgagtttggatacaagctgtgcatcttcgacagagacagcctgcctgggggaattgtcacagatgagaccctgagcttcattcagaaaagcagacgactcctggttgtcctaagtcccaactacgtgctccagggaacacaagccctcctggagctcaaggctggcctagaaaatatggcctcccggggcaacatcaacgtcattttagtgcagtacaaagctgtgaaggacatgaaggtgaaagagctgaagcgggctaagacggtgctcacggtcattaaatggaaaggagagaaatccaagtatcctcagggcaggttctggaagcagttgcaggtggccatgccagtgaagaagagtcccaggtggtctagcaatgacaagcagggtctctcctactcatccctgaaaaacgtatga

SEQ ID NO: 7 - DNA, 4576 bp, Artificial Sequence
  mRNA sequence of the humanized IL1RAP gene
ggggaggagctccggagggggaaaaagaaagtgcggcggaaagtaagaggctagtcggggaaggctgccagcatctggctttcctaggttcggcttggaccagggcgctgaaagaggcagtggtcggccaccctgcatccatctggtcgctgtcgccgagcctgctgggctggcccgataaggatgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaactggcttgtggttttggagccacagtctttctggtagtggttctcattgtggtttaccatgtttactggctggagatggtcctcttttaccgagctcactttggaacagatgaaacaattcttgatggaaaggagtatgatatttatgtttcctatgcaagaaatgtggaagaagaggaatttgtgctgctgacgctgcgtggagttttggagaatgagtttggatacaagctgtgcatcttcgacagagacagcctgcctgggggaattgtcacagatgagaccctgagcttcattcagaaaagcagacgactcctggttgtcctaagtcccaactacgtgctccagggaacacaagccctcctggagctcaaggctggcctagaaaatatggcctcccggggcaacatcaacgtcattttagtgcagtacaaagctgtgaaggacatgaaggtgaaagagctgaagcgggctaagacggtgctcacggtcattaaatggaaaggagagaaatccaagtatcctcagggcaggttctggaagcagttgcaggtggccatgccagtgaagaagagtcccaggtggtctagcaatgacaagcagggtctctcctactcatccctgaaaaacgtatgaaaggagaagtgagggggtacaaagaacaaggcggttcatgggaggaagggccccctctttccttctaggctgtggcttcatagacagaaaaagagtcctgtctaagccccctgggaggaaaatattccgggactgtacggctggcagctctgcctctcagcaatactaagcgttacagtgatggactccacagtccgccaccagttctaaaagtcattttgatctttacaagtaaagatgtgtctgatgcacccccacctctgttcttccctcttttctaatctcttcgtaaaaatctcacctctatggaactgctttgcccagtgactcaaaactctgtttaaagatactttacctgctgacaggagtatcgcctgtttctcagtaggacactcctttaattttctcttctcttccgtcatccattaagcatgtaatcagggactaaatctgtgtgagctgaggctatttcactcctttgcttctgtgtctgttgaatctaccacctaagcaagactaacagtcaaactgcagctgttcctcgtgactgattaagcgacatactgccttcctgctactaaaccccattcgctctcatctcaggtaatggagatcatatttataaacttgaaaaagccatggcttttgttttatcatagtaatttctcatctgtctacagatagacaggtcggtacttgttccttcctttccctacctattggatctgtcactcatatctcataggcagcatcacgcttgctgcggtctgcggggaagctttaatggtgctaatggagagtgagaaaatgcaaaagcgctctcagggcatttttcacatgggaagagagcacagaagagaatgcttggcggtttgtttgttaattctgaccctagaggggaaaaccaggaaagtggttaatcctcactccttcacacacctcttcattatgaagtggaggtgagaggtactgagtttccttttggcattactattccaaaagaatgattattcatggtaattaattgtgcagtgcctgtagtaccttgctccaaatggatcaccctgcaaatcattaataatgaagtggttttattagtttcaagcagaatttcactatttctcagacttgcaaaagtgaaaggcaagtataacattcacatttagagaggccaatttctatttcatatctaacatgtatatatatgtatatacatatatacatatatatatgatctctgtacacataaattttatatgtctatgtatatagaagtatccacaaatgacatgtgtgtgtgtatatccacctgcctataaattatctctgtgtattattttgggctatacataatcacacacatacatctttcagtagtcattggcaaaatattgaaagccagtagaactagtgttcaatctttaaattatgttaatagtgttatggttttgtgttgatattttcatttcatttcatttctgttccatttgtgaattcctggctctctgtatatagttgtcaaagttcttacctcccctaaattaatatgaacgacttaatgcttcaacagttgatctgggttgcaggggaatctcttgtttgtgaacctcccattgccactgctcaacagtgatcatctaatattgcttgagtttctactttattccctgaggtcacatttagaattgaagttaaactaagctatgtgaactgatgaagaaaaacagatattgttttaaagaagtctgagtaccctataaacatgactcataaattaaggaagaccttaaccaagttttgttaataatgttaagcaagtctaaaattacttattcatcagtactgaagtccacagttaaccattgctttgttagaattattgattactttaaattgaaaagagttcccttctgaatccttgacttaaaactatgatatagtgtcgttcttgcatctaaccttttagaagtgctaatgctgtttttggtatttcaaataattaccatttccactaataaccttttaatcacagtaccagataagttccacataaactttgaatgttaccttccgcatggacttatgggaacacaagggccagacttgtcaccacagctgttccctctgttctctgtagtttctgcacggttggagtctgtattcatcccagccactattatatcacctccatgtttttctatttataggcccttcttattctgagcccactctagtgctatagcataaagcatatcaaatctaaataacatatctcatcctaagtagatttaatttcaagactcagtgtttcagaagatgcttcaaatagaacagcagagagagggggacacacacttgtatttatgataatgagaacttctgcacccaggttaacaaagcagcatgctatgtagagtgtatatgtatacagccacatgggatttaattctcttttgtatcttagatgagtcccagagatgctaataaaaaaaaaagtactgtcttgtattgagaaacaaaaggatcactccacttgttaactacccaattgtttccataggttttagtactgtaagaccatttttaattttattcataaacatatgtattttccattttcatttgatttgtatatttacatatttttgcaataaaccgtttattttcacctttgaatttt

SEQ ID NO: 8 - protein, 570 aa, Artificial Sequence
  expressed amino acid sequence of the humanized IL1RAP gene
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVFLVVVLIVVYHVYWLEMVLFYRAHFGTDETILDGKEYDIYVSYARNVEEEEFVLLTLRGVLENEFGYKLCIFDRDSLPGGIVTDETLSFIQKSRRLLVVLSPNYVLQGTQALLELKAGLENMASRGNINVILVQYKAVKDMKVKELKRAKTVLTVIKWKGEKSKYPQGRFWKQLQVAMPVKKSPRWSSNDKQGLSYSSLKNV

SEQ ID NO: 9 - DNA, 228 bp, Artificial Sequence
  polyA (pA)
atcgataccgtcgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 10 - DNA, 98 bp, Artificial Sequence
  junction sequence between the 3′ end of the Neo cassette and the mouse sequence
ttcatcagtcaggtacataattaggtggatccactagttctagagcggccgcggacttctgtggtatttgatgagtctgtccttctatgggatcctgc

SEQ ID NO: 11 - DNA, 25 bp, Artificial Sequence
  primer
agggaggattgtgaggcaataccag

SEQ ID NO: 12 - DNA, 25 bp, Artificial Sequence
  primer
gttcaaagagtgggcacttgatgcg

SEQ ID NO: 13 - DNA, 20 bp, Artificial Sequence
  primer
gctcgactagagcttgcgga

SEQ ID NO: 14 - DNA, 25 bp, Artificial Sequence
  primer
tggagccaagacgtgttacgttagc

SEQ ID NO: 15 - DNA, 25 bp, Artificial Sequence
  primer
tcatggttgtctgtgagccagagga

SEQ ID NO: 16 - DNA, 25 bp, Artificial Sequence
  primer
agagaacgcaaatgcagaaagcagg

SEQ ID NO: 17 - DNA, 24 bp, Artificial Sequence
  primer
gtacacatgtgccacagggaaggt

SEQ ID NO: 18 - DNA, 25 bp, Artificial Sequence
  primer
ctctgggtcctggtggcactctatc

SEQ ID NO: 19 - DNA, 23 bp, Artificial Sequence
  primer
ggatcggccattgaacaagatgg

SEQ ID NO: 20 - DNA, 23 bp, Artificial Sequence
  primer
cagaagaactcgtcaagaaggcg

SEQ ID NO: 21 - DNA, 26 bp, Artificial Sequence
  primer
tgttggttctgacaataaagcttggg

SEQ ID NO: 22 - DNA, 25 bp, Artificial Sequence
  primer
cagaccccttcctaagttgtctagt

SEQ ID NO: 23 - DNA, 25 bp, Artificial Sequence
  primer
gttgagactcagaaagacgggcaat

SEQ ID NO: 24 - DNA, 25 bp, Artificial Sequence
  primer
gttcaaagagtgggcacttgatgcg

SEQ ID NO: 25 - DNA, 26 bp, Artificial Sequence
  primer
aggatcactccacttgttaactaccc

SEQ ID NO: 26 - DNA, 25 bp, Artificial Sequence
  primer
acatactcacccgaagcatgactct

SEQ ID NO: 27 - DNA, 25 bp, Artificial Sequence
  primer
gacaagcgttagtaggcacatatac

SEQ ID NO: 28 - DNA, 24 bp, Artificial Sequence
  primer
gctccaatttcccacaacattagt

SEQ ID NO: 29 - DNA, 20 bp, Artificial Sequence
  primer
cttccgcctcccagagaatc

SEQ ID NO: 30 - DNA, 20 bp, Artificial Sequence
  primer
ttccctggagcacgtagttg

SEQ ID NO: 31 - DNA, 22 bp, Artificial Sequence
  primer
cctgcaaagtgatgcctcagaa

SEQ ID NO: 32 - DNA, 21 bp, Artificial Sequence
  primer
cgtcagcagcacaaattcctc

SEQ ID NO: 33 - DNA, 22 bp, Artificial Sequence
  primer
tcaccatcttccaggagcgaga

SEQ ID NO: 34 - DNA, 21 bp, Artificial Sequence
  primer
gaaggccatgccagtgagctt

SEQ ID NO: 35 - DNA, 4393 bp, Artificial Sequence
  KI fragment
atgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaactggcttgtggttttggagccacagtctttctggtagtggttctcattgtggtttaccatgtttactggctggagatggtcctcttttaccgagctcactttggaacagatgaaacaattcttgatggaaaggagtatgatatttatgtttcctatgcaagaaatgtggaagaagaggaatttgtgctgctgacgctgcgtggagttttggagaatgagtttggatacaagctgtgcatcttcgacagagacagcctgcctgggggaattgtcacagatgagaccctgagcttcattcagaaaagcagacgactcctggttgtcctaagtcccaactacgtgctccagggaacacaagccctcctggagctcaaggctggcctagaaaatatggcctcccggggcaacatcaacgtcattttagtgcagtacaaagctgtgaaggacatgaaggtgaaagagctgaagcgggctaagacggtgctcacggtcattaaatggaaaggagagaaatccaagtatcctcagggcaggttctggaagcagttgcaggtggccatgccagtgaagaagagtcccaggtggtctagcaatgacaagcagggtctctcctactcatccctgaaaaacgtatgaaaggagaagtgagggggtacaaagaacaaggcggttcatgggaggaagggccccctctttccttctaggctgtggcttcatagacagaaaaagagtcctgtctaagccccctgggaggaaaatattccgggactgtacggctggcagctctgcctctcagcaatactaagcgttacagtgatggactccacagtccgccaccagttctaaaagtcattttgatctttacaagtaaagatgtgtctgatgcacccccacctctgttcttccctcttttctaatctcttcgtaaaaatctcacctctatggaactgctttgcccagtgactcaaaactctgtttaaagatactttacctgctgacaggagtatcgcctgtttctcagtaggacactcctttaattttctcttctcttccgtcatccattaagcatgtaatcagggactaaatctgtgtgagctgaggctatttcactcctttgcttctgtgtctgttgaatctaccacctaagcaagactaacagtcaaactgcagctgttcctcgtgactgattaagcgacatactgccttcctgctactaaaccccattcgctctcatctcaggtaatggagatcatatttataaacttgaaaaagccatggcttttgttttatcatagtaatttctcatctgtctacagatagacaggtcggtacttgttccttcctttccctacctattggatctgtcactcatatctcataggcagcatcacgcttgctgcggtctgcggggaagctttaatggtgctaatggagagtgagaaaatgcaaaagcgctctcagggcatttttcacatgggaagagagcacagaagagaatgcttggcggtttgtttgttaattctgaccctagaggggaaaaccaggaaagtggttaatcctcactccttcacacacctcttcattatgaagtggaggtgagaggtactgagtttccttttggcattactattccaaaagaatgattattcatggtaattaattgtgcagtgcctgtagtaccttgctccaaatggatcaccctgcaaatcattaataatgaagtggttttattagtttcaagcagaatttcactatttctcagacttgcaaaagtgaaaggcaagtataacattcacatttagagaggccaatttctatttcatatctaacatgtatatatatgtatatacatatatacatatatatatgatctctgtacacataaattttatatgtctatgtatatagaagtatccacaaatgacatgtgtgtgtgtatatccacctgcctataaattatctctgtgtattattttgggctatacataatcacacacatacatctttcagtagtcattggcaaaatattgaaagccagtagaactagtgttcaatctttaaattatgttaatagtgttatggttttgtgttgatattttcatttcatttcatttctgttccatttgtgaattcctggctctctgtatatagttgtcaaagttcttacctcccctaaattaatatgaacgacttaatgcttcaacagttgatctgggttgcaggggaatctcttgtttgtgaacctcccattgccactgctcaacagtgatcatctaatattgcttgagtttctactttattccctgaggtcacatttagaattgaagttaaactaagctatgtgaactgatgaagaaaaacagatattgttttaaagaagtctgagtaccctataaacatgactcataaattaaggaagaccttaaccaagttttgttaataatgttaagcaagtctaaaattacttattcatcagtactgaagtccacagttaaccattgctttgttagaattattgattactttaaattgaaaagagttcccttctgaatccttgacttaaaactatgatatagtgtcgttcttgcatctaaccttttagaagtgctaatgctgtttttggtatttcaaataattaccatttccactaataaccttttaatcacagtaccagataagttccacataaactttgaatgttaccttccgcatggacttatgggaacacaagggccagacttgtcaccacagctgttccctctgttctctgtagtttctgcacggttggagtctgtattcatcccagccactattatatcacctccatgtttttctatttataggcccttcttattctgagcccactctagtgctatagcataaagcatatcaaatctaaataacatatctcatcctaagtagatttaatttcaagactcagtgtttcagaagatgcttcaaatagaacagcagagagagggggacacacacttgtatttatgataatgagaacttctgcacccaggttaacaaagcagcatgctatgtagagtgtatatgtatacagccacatgggatttaattctcttttgtatcttagatgagtcccagagatgctaataaaaaaaaaagtactgtcttgtattgagaaacaaaaggatcactccacttgttaactacccaattgtttccataggttttagtactgtaagaccatttttaattttattcataaacatatgtattttccattttcatttgatttgtatatttacatatttttgcaataaaccgtttattttcacctttgaatttt

SEQ ID NO: 36 - protein, 570 aa, Rattus norvegicus
MGLPWCLMSLFFCGILQSHASERCDDWGLDTMRQIQVFEDEPARIKCPLFEHFLKYNYSTAHSSGLTLIWYWTRQDRDLEEPINFRLPENRISKEKDVLWFRPTLLNDTGNYTCMLRNTTYCSKVAFPLEVVQKDSCFNSPMRLPVHRLYIEQGIHNITCPNVDGYFPSSVKPSVTWYKGCTEIVNFHNVQPKGMNLSFFIPLVSNNGNYTCVVTYLENGRLFHLTRTMTVKVVGSPKDAVPPHIYSPNDRVVYEKEPGEELVIPCKVYFSFIMDSHNEIWWTIDGKKPDDVPVDITIIESVSYSSTEDETRTQILSIKKVTPEDLKRNYVCHARNAEGEAEQAAKVKQKVIPPRYTVELACGFGATVFLVVVLIVVYHVYWLEMVLFYRAHFGTDETILDGKEYDIYVSYARNAEEEEFVLLTLRGVLENEFGYKLCIFDRDSFPGGIVTDETLSFIQKSRRLLVVLSPNYVLQGTQALLELKAGLENMASRGNINVILVQYKAVKDLKVKELKRAKSVLTVIKWKGEKSKYPQGRFWKQLQVAMPVKKSPRWSSSDKQGLSYSSLKNV